(19) United States
(12) Patent Application Publication
(10) Pub. No.: US 2021/0069134 A1
(43) Pub. Date: Mar. 11, 2021

(54) COMPOSITIONS AND METHODS OF TREATING A SUBJECT WITH TAURINE AND DERIVATIVES THEREOF

(71) Applicant: The Research Foundation For SUNY, Albany, NY (US)

(72) Inventors: Lorenz Simon Neuwirth, Staten Island, NY (US); Bright U. Emenike, Jamesburg, NJ (US)

(73) Assignee: The Research Foundation for the State University of New York

(21) Appl. No.: 16/880,796

(22) Filed: May 21, 2020

Related U.S. Application Data
(60) Provisional application No. 62/851,472, filed on May 22, 2019.

(51) Int. Cl. A61K 31/185; A61P 39/02

(52) U.S. Cl. CPC A61K 31/185; A61K 9/20; A61P 39/02

(57) ABSTRACT

Disclosed are the methods and compositions for treating, ameliorating, or preventing neurological symptoms or conditions associated with lead (Pb²⁺) poisoning, and, also for reversing the damage caused by prolonged or acute lead (Pb²⁺) exposure. Compositions comprised of taurine or derivatives thereof, and optionally an injectable formulation, are also disclosed.

### CROSS-REFERENCES TO RELATES APPLICATIONS

This application claims priority benefit to U.S. Provisional Application No. 62/851,472 filed May 22, 2019, the contents of which are fully incorporated herein by reference.

### FIELD OF THE INVENTION

The present disclosure relates to compositions and methods of treating subjects in need thereof with taurine or taurine derivatives. For example, treating, ameliorating, or preventing one or more neurological symptoms or conditions associated with or caused by lead poisoning.

### BACKGROUND

Lead (Pb²⁺) is a metal typically found in the earth. However, certain activities such as burning fossil fuels and manufacturing have spread Pb²⁺ contamination throughout the world and into contact with animals and humans, resulting in environmental contamination and presenting public health problems, such as environmental lead (Pb²⁺) poisoning.

Pb²⁺ is a developmental neurotoxicant that causes Pb²⁺-induced frontoexecutive dysfunctions and lifelong cognitive dysfunction. Environmental Pb²⁺ poisoning causes brain damage in exposed children because of its neurotoxicity. Children and young adolescents are the most at risk for developmental neuropathologies and elevated levels of environmental Pb²⁺ exposure (i.e., ≥10 μg/dL in the U.S.) are considered a threat to the environment. Previously, brain damage caused by Pb²⁺ exposure was thought to be irreversible, but irreversible damage is selectively associated with high blood lead level (HBLLs) exposures (i.e., ≥39 μg/dL). At low blood lead level (LBLLs) exposures (i.e., ≤38 μg/dL or below ≤10 μg/dL in the U.S.), Pb²⁺'s neurotoxicant effects may be more susceptible during time-periods of neural plasticity and recovering from such injuries despite poisoning.

Pb²⁺ and other metal poisons have been primarily treated by chelation therapy to remove Pb²⁺ and/or other metals from the subject's blood stream. However, if a subject such as a child, cannot be removed from the source of the Pb²⁺ exposure or an acute exposure occurred at a dangerously high dose, the subject may experience high organ risk (i.e., injury and/or failure) from Pb²⁺ deposition, of which the brain is the most vulnerable organ to Pb²⁺ exposure at both HBLLs and LBLLS. Furthermore, even if Pb²⁺ is chelated from the blood stream, Pb²⁺ has the tendency to problematically mobilize and substitute for calcium (Ca²⁺) and ultimately deposit into bone stores. Thus, from a single Pb²⁺ exposure, long lasting risks for Pb²⁺ to re-mobilize back into the blood stream, from the cortical bones as well as the femur, can result in ongoing Pb²⁺ redistribution and neurotoxicity. Accordingly, subjects in need of treatment may problematically undergo frequent chelation therapy and blood transfusions if chelation therapy is unsuccessful.

Both high- and low-level exposures to environmental Pb²⁺ can cause a wide-range of developmental neuropathologies with varied behavioral and cognitive symptoms. Thus, although low-level Pb²⁺ exposures in the environment may improve living conditions according to public health standards; the same low-levels of Pb²⁺ exposure can significantly impact children's neurodevelopment in-utero and during critical periods from birth through the first few years of postnatal life. Thus, low-level Pb²⁺ exposure problematically remains both a challenge and a risk for children because trace metals are neurotoxicants regardless of exposure levels.

Although chelation therapy is an effective treatment for subjects that experience metal toxicity at high-level Pb²⁺ exposures (i.e., ≥39 μg/dL), chelation therapy may be inappropriate for lower levels of Pb²⁺ poisoning. Once Pb²⁺ deposits within the central and peripheral nervous system of a subject, the Pb²⁺ deposits are unable to be chelated and or filtered out of the blood, urine, or feces, unless the Pb²⁺ deposits are mobilized by Ca²⁺ transport systems or Ca²⁺-dependent second messenger systems. At present, beyond prescription metal chelators used to treat Pb²⁺, mercury (Hg²⁺), and arsenic (As⁻³) poisoning, there are no drugs currently available to specifically target the central and peripheral nervous tissues to support tissue and cell survival in the presence of metals that cannot be chelated.

Accordingly, what is needed is a drug to treat Pb²⁺ poisoning throughout the nervous system and compositions and methods of treating, ameliorating, or preventing one or more neurological symptoms or conditions associated with or caused by Pb²⁺ poisoning and/or reversing the damage caused by prolonged or acute Pb²⁺ exposure. Further, what is need are new therapies for subjects such as children that continue to face low-level Pb²⁺ exposures (i.e., ≤39 μg/dL). Moreover, therapies for neuroprotection are needed.

### SUMMARY

The present disclosure relates to compositions and methods of treating subjects in need thereof with taurine or taurine derivatives. In embodiments, the present disclosure relates to a method of treating, ameliorating, or preventing one or more neurological symptoms of Pb²⁺ poisoning in a subject having one or more neurological symptoms, including: administering a therapeutically effective amount of taurine or taurine derivative to a subject in need thereof.

In some embodiments, the present disclosure relates to a composition for treating, ameliorating, or preventing one or more neurological symptoms of Pb²⁺ poisoning in a subject, including: a compound including one or more of: 2-aminoethane-1-sulfonic acid, 3-aminopropanoic acid, 2-aminobenzenesulfonic acid, 2-(aminoethyl)phosphonic acid, 3-amino-N-(trifluoromethyl)propenamide, 3-amino N-hydroxypropanamide, 2-aminoethane-1-sulfinic acid, 3-aminopropane-1-sulfinic acid, 3-amino-3-fluoropanoic acid, 2-amino-2-fluoroethane-1-sulfinic acid, 3-amino-2-fluoropropane-1-sulfinic acid, 4-amino-3-fluorobutanoic acid, 3-amino-2-fluoropropanoic acid, 2-aminocyclopropane-1-carboxylic acid, or a pharmaceutically acceptable salt, hydrate or solvate thereof.

In some embodiments, the present disclosure relates to a pharmaceutical formulation, including: a compound selected from the group consisting of 2-aminoethane-1-sulfonic acid, 3-aminopropanoic acid, 2-aminobenzenesulfonic acid, 2-(aminoethyl)phosphonic acid, 3-amino-N-(trifluoromethyl)propenamide, 3-amino N-hydroxypropanamide, 2-aminoethane-1-sulfinic acid, 3-aminopropane-1-sulfinic acid, 3-amino-3-fluoropanoic acid, 2-amino-2-fluoroethane-1-sulfinic acid, 3-amino-2-fluoropropane-1-sulfinic acid, 4-amino-3-fluorobutanoic acid, 3-amino-2-fluoropropanoic acid, 2-aminocyclopropane-1-carboxylic acid, or a pharmaceutically acceptable salt, hydrate or solvate thereof; and a pharmaceutically acceptable vehicle. In embodiments, the compound is present in an amount sufficient to bind to one or more gamma amino butyric acid (GABA-_{A}) receptors, one or more n-methyl-D-aspartate (NMDA) receptors, or one or more glycine (Gly) receptors disposed within a subject.

The illustrative aspects of the present disclosure are designed to solve the problems herein described and/or other problems not discussed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features of this disclosure will be more readily understood from the following detailed description of the various aspects of the disclosure taken in conjunction with the accompanying drawings that depict various embodiments of the disclosure, in which:

FIG. 1 depicts a Liquid Chromatography/Mass Spectroscopy (LC/MS) detection profile of standards for neurotransmitters as described below.

FIG. 2A and FIG. 2B are histograms depicting differences in male rats' ability to learn odor (OD) and digging medium (MD) simple discriminations as described below.

FIG. 3A and FIG. 3B are histograms depicting differences in female rats' ability to learn odor (OD) and digging medium (MD) simple discriminations as described below.

FIG. 4 is a graph depicting a rate-of-learning cumulative records for a single representative male rat from the Control (upper panel), Perinatal (middle panel), and Perinatal+Taurine (lower panel) treatment groups described below.

FIG. 5 is a graph depicting the rate-of-learning cumulative records for a single representative female rat from the Control (upper panel), Perinatal (middle panel), and Perinatal+Taurine (lower panel) treatment groups described below.

FIG. 6A and FIG. 6B are histograms depicting the male rat reacquisition learning data between test days to ensure their behavioral momentum as described below.

FIG. 7A and FIG. 7B are histograms depicting the female rat reacquisition learning data between test days to ensure their behavioral momentum as described below.

FIG. 8A and FIG. 8B are histograms depicting the male rats ASST performance for TTC and ETC as described below.

FIGS. 9A and 9B are histograms depicting the female rats ASST performance as described below.

FIGS. 10A, 10B, 10C, and 10D are histograms depicting the male rats LC/MS GABA:Neurotransmitter ratios as described below.

FIGS. 11A, 11B, 11C, and 11D are histograms depicting the female rats LC/MS GABA:Neurotransmitter ratios as described below.

FIGS. 12A, 12B, 12C, and 12D are histograms the male rats LC/MS Taurine:Neurotransmitter ratios as described below.

FIGS. 13A, 13B, 13C, and 13D are histograms the female rats LC/MS Taurine:Neurotransmitter ratios as described below.

FIGS. 14A, 14B, 14C, and 14D are histograms depicting male and female rats LC/MS GABA:Neurotransmitter and Taurine:Neurotransmitter ratios as described below.

FIG. 15 depicts chemical structures for taurine and taurine derivatives of the present disclosure.

FIGS. 16A and 16B are graphs relating to the preliminary assessment of rat locomotor activity as described below.

FIGS. 17A, 17B, 17C, and 17D are graphs depicting an assessment of Pb²⁺-exposure on rat locomotor activity as described below.

FIGS. 18A and 18B are histograms relating to rats subjected to the EPM.

FIG. 19 depicts a rat track plot from each treatment condition and their group mean activity average across the 10-min EPM test for male rats.

FIG. 20 depicts a rat track plot from each treatment condition and their group mean activity average across the 10-min EPM test for female rats.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

The present disclosure relates to compositions and methods for application of taurine or taurine derivatives to subjects in need thereof. The method includes administering a predetermined amount of taurine or taurine derivatives to a subject in need thereof such as a therapeutic effective amount. A treatment in accordance with the present disclosure includes treating subjects in need thereof with taurine or taurine derivatives to treat, ameliorate, or prevent one or more neurological symptoms of Pb²⁺ poisoning in a subject such as anxiety or loss in cognitive function induced by Pb²⁺ poisoning. Further, compositions and methods of the present disclosure counteract neurotoxicant Pb²⁺ exposures, and prophylactically prevent brain injury. Taurine and taurine derivative therapy as described herein is beneficial in that it is a cost-effective drug treatment option for individuals who come from low social economic status. Further, taurine and taurine derivatives have the unique ability to serve a dual function as both an anxiolytic and nootropic neuromodulatory compound that can regulate imbalances in the neurochemistry of individuals with intellectual disabilities, anxiety and affective disorders that arise from aberrant neurodevelopment. As such, in embodiments, the present disclosure provides the benefit of a single drug for psychopharmacotherapeutic interventions that would otherwise require a mixed drug cocktail. This substantially reduces the concerns for undesirable drug side-effects and reduces the drug-to-drug interactions that might also occur when prescribing cocktails. Benefits of embodiments of the present disclosure also include subject recovery from neurodevelopmental disorders induced by environmentally relevant (e.g., ≤5-10 μg/dL BLL poisoning). Further, taurine and taurine derivatives beneficially act as neuroprotective agents and ameliorate behavioral, affective, and cognitive symptoms emanating from neurotoxicants.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein the terms “drug,” “drug substance,” “active pharmaceutical ingredient,” and the like, refer to a compound (e.g., taurine or taurine derivative) that may be used for treating a subject in need of treatment.

As used herein the term “excipient” or “adjuvant” refers to any inert substance.

As used herein the terms “drug product,” “pharmaceutical dosage form,” “dosage form,” “final dosage form” and the like, refer to a pharmaceutical composition that is administered to a subject in need of treatment and generally may be in the form of tablets, capsules, sachets containing powder or granules, liquid solutions or suspensions, patches, and the like.

As used herein the term “solvate” describes a molecular complex including the drug substance (e.g., taurine and taurine derivatives) and a stoichiometric or non-stoichiometric amount of one or more pharmaceutically acceptable solvent molecules.

The term “hydrate” describes a solvate including the drug substance and a stoichiometric or non-stoichiometric amount of water.

As used herein the term “pharmaceutically acceptable” substances refers to those substances which are within the scope of sound medical judgment suitable for use in contact with the tissues of subjects without undue toxicity, irritation, allergic response, and the like, and effective for their intended use.

As used herein the term “pharmaceutical composition” refers to the combination of one or more drug substances and one or more excipients such as taurine or one or more taurine derivatives and one or more pharmaceutically acceptable vehicles with which the one or more taurine or taurine derivatives is administered to a subject.

As used herein, the term “pharmaceutically acceptable salt” refers to a salt of a compound, which possesses the desired pharmacological activity of the parent compound. Non-limiting examples of pharmaceutically acceptable salts include: acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, for example, an alkali metal ion, an alkaline earth ion, or an aluminum ion; or coordinates with an organic base such as ethanolamine, diethanolamine, triethanolamine, N-methylglucamine, and the like.

As used herein the term “pharmaceutically acceptable vehicle” refers to a diluent, adjuvant, excipient or carrier with which a compound is administered.

As used herein the term “prevent”, “preventing” and “prevention” of neurological symptoms of Pb²⁺ poisoning means (1) reducing the risk of a patient who is not experiencing neurological symptoms of Pb²⁺ poisoning from developing neurological symptoms of Pb²⁺ poisoning, or (2) reducing the frequency of, the severity of, or a complete elimination of, neurological symptoms of Pb²⁺ poisoning already being experienced by a subject.

As used herein the term “subject” includes humans, animals or mammals. The terms “subject” and “patient” may be used interchangeably herein.

As used herein the term “therapeutically effective amount” means the amount of a compound that, when administered to a subject for treating or preventing neurological symptoms of Pb²⁺ poisoning, is sufficient to effect such treatment or prevention of the neurological symptoms of Pb²⁺ poisoning. A “therapeutically effective amount” can vary depending, for example, on the compound, the severity of the neurological symptoms of Pb²⁺ poisoning, the etiology of the neurological symptoms of Pb²⁺ poisoning, the age of the subject to be treated and/or the weight of the subject to be treated. A “therapeutically effective amount” is an amount sufficient to alter the subjects' natural state.

As used herein the term “treat”, “treating” and “treatment” of neurological symptoms of Pb²⁺ poisoning means reducing the frequency of symptoms of neurological symptoms of Pb²⁺ poisoning, eliminating the symptoms of neurological symptoms of Pb²⁺ poisoning, avoiding or arresting the development of neurological symptoms of Pb²⁺ poisoning, ameliorating or curing an existing or undesirable neurological symptom caused by environmental Pb²⁺ exposure, and/or reducing the severity of symptoms of neurological symptoms of Pb²⁺ poisoning.

“Retained in the stomach,” when used in connection with a pharmaceutical composition or dosage form, means that at least a portion of the dosage form remains in a subject's stomach following oral administration for about three or more hours.

“Release,” “released,” and the like, when used in connection with a pharmaceutical composition or dosage form, refers to the portion of the drug substance that leaves the dosage form following contact with an aqueous environment.

As used herein, when any variable occurs more than one time in a chemical formula, its definition on each occurrence is independent of its definition at every other occurrence.

### DESCRIPTION OF CERTAIN EMBODIMENTS

The present disclosure relates to a method of treating, ameliorating, or preventing one or more neurological symptoms of Pb²⁺ poisoning in a subject having one or more neurological symptoms, including: administering a therapeutically effective amount of taurine or taurine derivative to a subject in need thereof. In embodiments, the present disclosure uses taurine (2-amino ethanesulfonic acid), and derivatives thereof, to beneficially counteract against the neurotoxicant Pb²⁺. In embodiments, the compositions and methods of the present disclosure beneficially treat, ameliorate, prevent, or reduce symptoms associated with and/or caused by developmental Pb²⁺ poisoning, which, dependent upon the time-period and the acute versus chronic duration of exposure, causes a range of intellectual cognitive, affective, and behavioral disorders that can be recovered by taurine and taurine derivative psychopharmacotherapy as described herein. The compositions and methods of the present disclosure beneficially provide a drug to treat Pb²⁺ poisoning throughout the nervous system and methods of treating, ameliorating, or preventing one or more neurological symptoms or conditions associated with or caused by Pb²⁺ poisoning and reversing the damage caused by prolonged or acute Pb²⁺ exposure. Further, therapies for subjects such as children that continue to face low-level Pb²⁺ exposures (i.e., ≤39 μg/dL or between 0.5 μg/dL to 38 μg/dL) are provided along with therapies for neuroprotection.

In embodiments, the present disclosure combats Pb²⁺ toxicity in subjects in need thereof using taurine (2-aminoethanesulfonic acid) and/or functional derivatives thereof. In embodiments, taurine and taurine derivatives are useful for counteracting neurotoxicant Pb²⁺ exposures, and for recovering losses in cognitive function induced by Pb²⁺ poisoning. In embodiments, taurine or taurine derivative treatment in accordance with the present disclosure ameliorates symptoms to levels comparable with subjects that were not exposed to any Pb²⁺. In embodiments, taurine or taurine derivative treatment in accordance with the present disclosure ameliorates symptoms such as anxiety and loss of cognitive function to levels at least 10%, at least 25%, at least 50%, or between 10% and 95% improved compared to the subject's initial presentation for treatment. Moreover, taurine or taurine derivative treatment in accordance with the present disclosure can be tailored based on gender and level of exposure, to maximize efficacy. To further increase efficacy, taurine or taurine derivative treatment in accordance with the present disclosure can be manufactured as time-release dosage forms such as tablets and capsules to meet the specific needs of patients based on their clinical profiles and unique symptomology.

In embodiments, method for using taurine, and taurine derivatives to ameliorate the ill-effects of lead toxicity are disclosed. Non-limiting examples of ill-effects include anxiety, loss of affection, and loss of cognitive processing, as well as the associated social-emotional processing issues that underlie value for goal-directed behaviors and motivational factors in which behaviors manifest. In embodiments, taurine treatment does not actually remove Pb²⁺ from the bloodstream, or otherwise reduce blood levels. Rather, taurine and taurine derivative treatment in accordance with the present disclosure exhibits anxiolytic and nootropic properties, which counteract the adverse symptoms of lead-toxicity or prolonged exposure to Pb²⁺ (at both high- and low-levels of exposure during neural development). As a psychopharmacological medical intervention for Pb²⁺ toxicity, taurine treatment is unique because it combats the symptoms rather than the cause of the symptoms. For example, in subjects with reduced working memory due to Pb²⁺ exposure, after taurine treatment had been administered, subjects perform very close to control groups that had no Pb²⁺ exposure. In embodiments, taurine and taurine derivatives are anxiolytic such that taurine and taurine derivatives treatment reduced anxiety and anxiety-like behaviors, while, as a nootropic, taurine and taurine derivatives treatment increased frontoexecutive functions, particularly learning and remembering, which correlated with an overall increase in recovering/sustaining intelligence. In embodiments, frontoexecutive functions are increased by 1-20%, such as 5-15%. In embodiments, anxiety and anxiety-like behaviors are eliminated or reduced by 50 to 95%.

In embodiments, taurine is characterized as an organic compound known as 2-aminoethanesulfonic acid. In embodiments, taurine has a molecular formula C₂H₇NO₃S and a molecular weight of 125.1. In some embodiments, the drug or compound suitable for use in accordance with the present disclosure is a derivative of taurine such as one or more of 3-aminopropanoic acid, 2-aminobenzenesulfonic acid, 2-(aminoethyl)phosphonic acid, 3-amino-N-(trifluoromethyl)propenamide, 3-amino N-hydroxypropanamide, 2-aminoethane-1-sulfinic acid, 3-aminopropane-1-sulfinic acid, 3-amino-3-fluoropanoic acid, 2-amino-2-fluoroethane-1-sulfinic acid, 3-amino-2-fluoropropane-1-sulfinic acid, 4-amino-3-fluorobutanoic acid, 3-amino-2-fluoropropanoic acid, 2-aminocyclopropane-1-carboxylic acid, and combinations thereof.

In embodiments, methods of making taurine and taurine derivatives are known in the art.

In some embodiments, examples of taurine and taurine derivative are compounds chosen from one or more of formula (1) to formula (14):

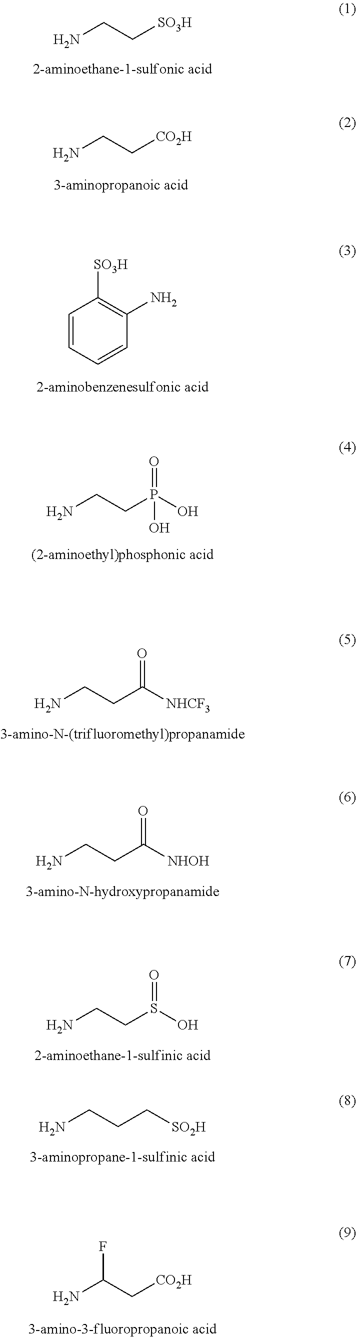

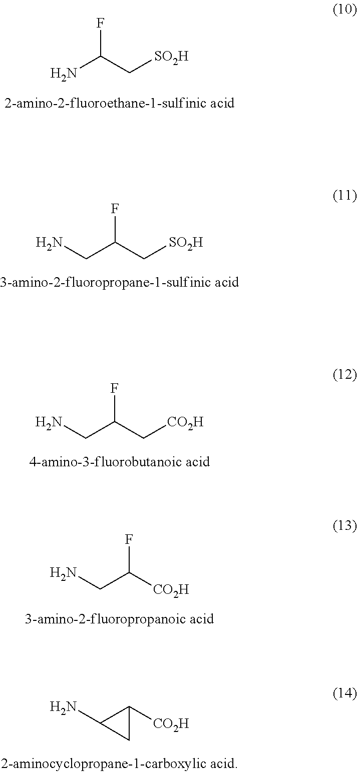

In some embodiments, taurine and taurine derivatives include a pharmaceutically acceptable salt, hydrate or solvate of compounds 1-14 shown above.

In embodiments, the amount of taurine or taurine derivative that will be effective in the treatment of one or more neurological symptoms of Pb²⁺ poisoning in a patient can depend on, among other factors, the specific amount of Pb²⁺ poisoning (e.g., chronic or acute depending upon amount and duration of exposure to environmental Pb²⁺), the subject being treated (e.g., fetus, child, or pregnant mother), the weight of the subject, the severity of the neurological symptom (e.g., anxiety or loss of cognitive functions) condition which is causing the Pb²⁺ exposure, the manner of administration, the formulation and the judgment of the prescribing physician. In embodiments, the amount of taurine and taurine derivative that will be effective in the treatment of the one or more neurological symptoms of lead poisoning in in a patient can be determined by standard clinical techniques known in the art. In addition, in-vitro or in-vivo assays may be employed to identify optimal dosage ranges. Oral compositions of the present disclosure can be adapted to be administered to a patient no more than twice per day, and in certain embodiments, only once per day. When a composition of the present disclosure is administered using an extended release delivery system, the dosing can be no more than once per day, and in certain embodiments, less than 3 times per week. Dosing may be provided alone or in combination with other drugs and treatments such as chelation and may continue as long as required for effective treatment of the one or more neurological symptoms.

In embodiments, suitable dosage ranges for administration can depend on the potency of the particular taurine or taurine derivative and the area of brain or brain receptor that is suitable for alleviating the one or more neurological symptoms. In certain embodiments, a therapeutically effective dose for treating one or more neurological symptoms such as anxiety or loss of cognitive function can range from about 0.05 mg to about 200 mg of taurine or taurine derivative per kilogram of subject per day, and in certain embodiments from about 0.05 mg to about 200 mg per kilogram of the subject per day. Dosage ranges may be readily determined by methods known to the skilled artisan. In embodiments, the taurine or taurine derivative is administered through interperitoneal injection in quantities less than 43 mg/kg/day or through a second route of administration at equivalent physiological dosage. In embodiments, the taurine or taurine derivative is administered in a drinking water solution containing both Pb²⁺ and taurine or taurine derivative, wherein the taurine or taurine derivative is present at about 0.05% of the total drinking water. In some embodiments, the taurine or taurine derivative is administered during gestational, perinatal, and early postnatal development of the subject, and wherein the subject is exposed to Pb²⁺. In embodiments, the taurine or taurine derivative is administered upon early maturation of the subject, for example early maturation may refer to a child 7 to 12 years old, and extend up until the 25 years of age when the brain's prefrontal cortex that governs fronto-executive functions fully matures. In embodiments, the taurine or taurine derivative is administered through interperitoneal injection in quantities less than 43 mg/kg or through a second route of administration at equivalent physiological dosage.

In embodiments, the concentration of taurine and taurine derivative in a composition, such as an extended release pill or injectable composition, can vary a great deal, and will depend on a variety of factors, including the type and severity of one or more neurological symptoms of lead poisoning, the desired duration of relief from one or more neurological symptoms of lead poisoning, possible adverse reactions, the effectiveness of the taurine or taurine derivative, and other factors within the particular knowledge of the patient and physician. In certain embodiments, compositions of the present disclosure can include an amount taurine or taurine derivative ranging from about 0.5 percent weight (wt %) to about 50 wt % of the total composition, in certain embodiments from about 0.5 wt % to about 5 wt % or the total composition, and in certain embodiments from about 5 wt % to about 20 wt % of the total composition.

Methods of treating or preventing one or more neurological symptoms of Pb²⁺ poisoning of the present disclosure can include administering to the subject a therapeutically effective amount of a taurine or taurine derivative to a patient in need of such treatment. A taurine or taurine derivative, or a pharmaceutical composition containing same, can be administered orally or intraperitoneally to the subject. Oral administration of a taurine or taurine derivative to a subject includes administering an oral composition of the present disclosure such as an extended release pill.

In embodiments, one dosage form suitable for administration of taurine and taurine derivatives includes compositions such as a delayed release capsule or pill. In embodiments, the amount of taurine or taurine derivative in a in a typical composition of the present disclosure can range from about 1 wt % to about 25 wt % of the total composition, such as about 5 wt % to 10 wt % of the total composition.

In embodiments, in addition to the taurine and taurine derivative, the pharmaceutical composition includes various excipients, such as a matrix forming agent and a swelling agent. In embodiments, such as tablets, the matrix forming agent provides structural integrity and helps control or extend the rate of drug release, among other functions. In embodiments, the matrix forming agent may include about 5% to about 45% of the pharmaceutical composition by weight and often includes about 20% to about 35% of the pharmaceutical composition by weight. Non-limiting examples of matrix forming agents are known in the art and examples may include those described in U.S. Patent Publication No. 20140163103 (herein entirely incorporated by reference). In embodiments, the pharmaceutical composition may include other excipients, including a swelling agent. In embodiments, the swelling agent may comprise about 5% to about 70% of the pharmaceutical composition by weight, or about 20% to about 55% of the pharmaceutical composition by weight, or about 30% to about 55% of the pharmaceutical composition by weight.

In embodiments, to prepare the drug product, the components of the pharmaceutical composition are blended and fabricated by methods known in the art. The resulting mixture is subsequently compacted in a press to yield individual (unit) dosages (tablets or capsules). To prepare the final drug product, the compressed dosage forms may undergo further processing, such as polishing, coating, and the like. In embodiments, the dosage form is configured to be retained in the stomach for several hours such as 3-6 hours and releases taurine or taurine derivative over an extended period of time such as 5-20 hours, or 5-10 hours.

In embodiments, non-limiting examples of suitable dosage forms include injectable dosage forms where taurine and taurine derivatives are dissolved in a delivery vehicle such as water. In some embodiments, the taurine or taurine derivative is disposed within a pharmaceutically acceptable vehicle. In embodiments, the taurine or taurine derivative is administered in an extended release pill. In embodiments, the taurine or taurine derivative is administered intraperitoneal injection.

In embodiments, the taurine or taurine derivative suitable for use herein has a binding affinity sufficient to bind to one or more gamma amino butyric acid (GABA-_{A}) receptors, or one or more gamma amino butyric acid (GABA-_{A}) receptors subunit configurations. In embodiments, the taurine or taurine derivative has a binding affinity sufficient to bind to one or more glycine (Gly) receptors, or one or more glycine (Gly) receptors subunit configurations. In embodiments, the taurine or taurine derivative has a binding affinity sufficient to bind to one or more n-methyl-D-aspartate (NMDA) receptors, or one or more n-methyl-D-aspartate (NMDA) receptors subunit configurations. In embodiments, the taurine or taurine derivative has a binding affinity sufficient to bind to one or more n-methyl-D-aspartate (NMDA) receptor subunits or subunit configurations at one or more glycine binding sites. In embodiments, taurine or taurine derivative binding affinity is sufficient to bind to one or more gamma amino butyric acid (GABA-_{A}) receptors, or one or more gamma amino butyric acid (GABA-_{A}) receptors subunit configurations, one or more n-methyl-D-aspartate (NMDA) receptor subunits or subunit configurations, and/or one or more glycine (Gly) receptors, or one or more glycine (Gly) receptors subunit configurations and change the state of the subject to treat, ameliorate, or prevent one or more neurological symptoms of lead poisoning in a subject. Non-limiting examples of neurological symptoms include anxiety, panic, affected disorder, and cognitive loss or deficiency.

In embodiments, the present disclosure relates to a method of treating, ameliorating, or preventing one or more neurological symptoms of Pb²⁺ poisoning in a subject having one or more neurological symptoms, comprising: administering a therapeutically effective amount of taurine or taurine derivative to a subject in need thereof, wherein the subject is a pregnant female mammal including a fetus, wherein the therapeutically effective amount is an amount sufficient for neuroprotection of the fetus from contact with Pb²⁺.

In embodiments, the present disclosure relates to a method of treating, ameliorating, or preventing one or more neurological symptoms of Pb²⁺ poisoning in a subject having one or more neurological symptoms, comprising: administering a therapeutically effective amount of taurine or taurine derivative to a subject in need thereof, wherein the therapeutically effective amount is an amount sufficient for neuroprotection of the child from contact with Pb²⁺.

In some embodiments, the present disclosure relates to a method of treating, ameliorating, or preventing one or more neurological symptoms of lead (Pb²⁺) poisoning in a subject having one or more neurological symptoms, comprising: administering a therapeutically effective amount of taurine or taurine derivative to a subject in need thereof. In some embodiments, the taurine or taurine derivative has a binding affinity sufficient to bind to one or more gamma amino butyric acid (GABA-_{A}) receptors, or one or more gamma amino butyric acid (GABA-_{A}) receptors subunit configurations. In some embodiments, the taurine or taurine derivative has a binding affinity sufficient to bind to one or more glycine (Gly) receptors, or one or more glycine (Gly) receptors subunit configurations. In some embodiments, the taurine or taurine derivative has a binding affinity sufficient to bind to one or more n-methyl-D-aspartate (NMDA) receptors, or one or more n-methyl-D-aspartate (NMDA) receptors subunit configurations. In some embodiments, the subject comprises one or more n-methyl-D-aspartate (NMDA) receptors, wherein the taurine or taurine derivative has a binding affinity sufficient to bind the taurine or taurine derivative to the one or more n-methyl-D-aspartate (NMDA) receptor subunit configurations at one or more glycine binding sites. In some embodiments, the taurine derivative is selected from the group consisting of a compound selected from the group consisting of 3-aminopropanoic acid, 2-aminobenzenesulfonic acid, 2-(aminoethyl)phosphonic acid, 3-amino-N-(trifluoromethyl)propenamide, 3-amino N-hydroxypropanamide, 2-aminoethane-1-sulfinic acid, 3-aminopropane-1-sulfinic acid, 3-amino-3-fluoropanoic acid, 2-amino-2-fluoroethane-1-sulfinic acid, 3-amino-2-fluoropropane-1-sulfinic acid, 4-amino-3-fluorobutanoic acid, 3-amino-2-fluoropropanoic acid, 2-aminocyclopropane-1-carboxylic acid, and combinations thereof. In some embodiments, the taurine or taurine derivative is a pharmaceutically acceptable salt, hydrate or solvate thereof. In some embodiments, the taurine or taurine derivative is disposed within a pharmaceutically acceptable vehicle. In some embodiments, the taurine or taurine derivative is administered during gestational, perinatal, and early postnatal development of the subject, and wherein the subject is exposed to Pb²⁺. In some embodiments, the taurine or taurine derivative is administered upon early maturation of the subject. In some embodiments, the taurine or taurine derivative is administered through interperitoneal injection in quantities less than 43 mg/kg or through a second route of administration at equivalent physiological dosage. In some embodiments, the taurine or taurine derivative is administered in a drinking water solution containing both Pb²⁺ and taurine or taurine derivative, wherein the taurine or taurine derivative is present at about 0.05% of the total drinking water solution. In some embodiments, the taurine or taurine derivative is administered in an extended release pill. In some embodiments, the taurine or taurine derivative is administered intraperitoneal injection. In some embodiments, the subject is a pregnant female mammal comprising a fetus, wherein the therapeutically effective amount is an amount sufficient for neuroprotection of the fetus from contact with Pb²⁺. In some embodiments, the subject is a developing child, wherein the therapeutically effective amount is an amount sufficient for neuroprotection of the child from contact with lead (Pb²⁺).

In some embodiments, the present disclosure relates to a composition for treating, ameliorating, or preventing one or more neurological symptoms of Pb²⁺ poisoning in a subject, including: a compound including one or more of: 2-aminoethane-1-sulfonic acid, 3-aminopropanoic acid, 2-aminobenzenesulfonic acid, 2-(aminoethyl)phosphonic acid, 3-amino-N-(trifluoromethyl)propenamide, 3-amino N-hydroxypropanamide, 2-aminoethane-1-sulfinic acid, 3-aminopropane-1-sulfinic acid, 3-amino-3-fluoropanoic acid, 2-amino-2-fluoroethane-1-sulfinic acid, 3-amino-2-fluoropropane-1-sulfinic acid, 4-amino-3-fluorobutanoic acid, 3-amino-2-fluoropropanoic acid, 2-aminocyclopropane-1-carboxylic acid, or a pharmaceutically acceptable salt, hydrate or solvate thereof. In some embodiments, the composition is disposed within a formulation comprising a pharmaceutically acceptable vehicle. In some embodiments, the formulation is an extended release composition or injectable solution. In some embodiments, the neurological symptom is anxiety, decreased cognitive function, or combinations thereof. In some embodiments, the subject is a mammal.

In some embodiments, the present disclosure relates to a pharmaceutical formulation, including: a compound selected from the group consisting of 2-aminoethane-1-sulfonic acid, 3-aminopropanoic acid, 2-aminobenzenesulfonic acid, 2-(aminoethyl)phosphonic acid, 3-amino-N-(trifluoromethyl)propenamide, 3-amino N-hydroxypropanamide, 2-aminoethane-1-sulfinic acid, 3-aminopropane-1-sulfinic acid, 3-amino-3-fluoropanoic acid, 2-amino-2-fluoroethane-1-sulfinic acid, 3-amino-2-fluoropropane-1-sulfinic acid, 4-amino-3-fluorobutanoic acid, 3-amino-2-fluoropropanoic acid, 2-aminocyclopropane-1-carboxylic acid, or a pharmaceutically acceptable salt, hydrate or solvate thereof; and a pharmaceutically acceptable vehicle, wherein the compound is present in an amount sufficient to bind to one or more GABA-_{A} receptors, one or more NMDA receptors, or one or more Gly receptors disposed within a subject.

### EXAMPLES

The following examples describe in detail preparation of compounds and compositions disclosed herein and assays for using compounds and compositions disclosed herein. It will be apparent to those of ordinary skill in the art that many modifications, both to materials and methods, may be practiced.

### Example 1—Early Neurodevelopmental Exposure to Low Lead Levels Induces Fronto-Executive Dysfunctions that are Recovered by Taurine Co-Treatment in the Rat Attention-Set Shift Test: Implications for Taurine as a Psychopharmacotherapy Against Neurotoxicants

The effects of developmental Pb²⁺ exposure (150 ppm lead acetate in drinking water) in Long Evans Hooded rats through the Attention Set-Shift Test (ASST) between postnatal days (PND) 60-90. Treatment groups were comprised of Control (0 ppm), Perinatal (150 ppm), and Perinatal+Taurine (150 ppm+0.05% Taurine in the drinking water) rats (N=36; n=6 per treatment group for each sex). Frontoexecutive functions were evaluated based on trials-to-criterion (TTC) and errors-to-criterion (ETC) measures for simple and complex discriminations (SD & CD), intradimensional and extradimensional shifts (ID & ED), as well as reversals of the CD-Rev, ID-Rev, and ED-Rev stages, respectively. Post-testing, the prelimbic (PrL), infralimbic (IL), orbital ventral frontal (OV), orbital ventro-lateral (OVL), and hippocampal (HP) brain regions were extracted and processed through Liquid Chromatography/Mass Spectroscopy (LC/MS) for determining the GABA and Taurine ratios relative to Glutamate, Dopamine, Norepinephrine, Epinephrine, and Serotonin. The ASST data revealed that Perinatal rats are negatively impacted by developmental Pb²⁺ exposures evidenced by increased TTC and ETC to learn the SD, ID, and ID-Rev with unique sex-based differences in frontoexecutive dysfunctions. Moreover, Perinatal+Taurine co-treated rats recovered these frontoexecutive dysfunctions to levels equivalent to Control rats. The LC/MS data revealed region specific patterns across the PrL, IL, OV, OVL, and HP in response to developmental Pb²⁺-exposure that produced an altered neurochemical signaling profile in a sex-dependent manner, which may underlie the observed frontoexecutive dysfunctions, cognitive inflexibility, and associated motivation deficits. When taurine co-treatment was administered concurrently for the duration of developmental Pb²⁺-exposure, the observed frontoexecutive dysfunctions were significantly reduced in both ASST task performance and neurochemical ratios that were comparable to Control levels for both sexes. Altogether, the data suggest that taurine co-treatment facilitates neuroprotection, mitigates neurotransmitter excitability balancing, and ameliorates against neurotoxicant exposures in early development as a potential psychopharmacotherapy.

### Methods

Subjects

In accordance with The SUNY Old Westbury (SUNY-OW) IACUC approval guidelines, Long-Evans Norwegian hooded male (N=3) and female rats (N=6) (Taconic, N.J.) were paired for breeding and their male and female F1 generation offspring were used for the present study. Rat litters were culled to 8-10 pups in order to control for maternal social influences on neurodevelopmental and behavioral outcomes that were later examined. Rats were randomly assigned to the following breeding groups: Control, Perinatal, or Perinatal+Taurine exposures, respectively. All rats were fed regularly with Purina rat chow (RHM1000 #5P07) ad libitum. However, Control rats were provided with regular water, while the experimental rats were fed water containing Pb²⁺ acetate (Sigma Aldrich, St. Louis, Mo.) from pairing throughout gestation and continued through weaning at postnatal day (PND) 22 (i.e., constituting a Perinatal develop-mental Pb²⁺ exposure model). At PND 22, Pb²⁺ exposures ceased and all rats returned to a regular water regimen. Rats assigned to the Perinatal group drank a lead acetate water (C₂H₃O₂)₂Pb.3H₂O [363.83 μM] and the Perinatal+Taurine group drank the identical lead acetate water, but it was additionally supplemented with 0.05% Taurine C₂H₇NO₃S₁ [4 mM] (Sigma Aldrich, St. Louis, Mo.). All water solutions were administered ad libitum. Prior to behavioral testing, all rats were handled for 20-min per day for 2-weeks. Between postnatal days (PNDs) 60-90 (i.e., when the prefrontal cortex is fully matured in rats) male and female rats were randomly selected from the litters and then assigned to the ASST. The following samples sizes were used within the ASST: n=6 Control,n=6 Perinatal, and n=6 Perinatal+Taurine for both males and females, respectively.

Blood Lead Level Analyses

At PND 22 immediately following the end of Pb²⁺-exposure, a separate group of male and female rats (i.e., with a representative sample culled from the same litters) were sacrificed (n=4 per gender, per treatment group) and their blood samples were collected and analyzed consistent with previous reports (Neuwirth, 2014; Neuwirth et al., 2017, Neuwirth et al., 2018b; Neuwirth et al., 2019a; Neuwirth et al., 2019b). Briefly, blood samples were collected within 2 mL anti-coagulant ethyenediaminetetraacefic acid (EDTA) coated syringes (Sardstedt, Germany), mixed to prevent coagulation, and then frozen at −80° C. Blood samples were analyzed using a commercial ESA LeadCare II Blood Lead Analyzer system (Magellan Diagnostics, North Billerica, Mass.) to determine the amount of Pb²⁺ in the blood by electrochemical anodic stripping voltammetry (ASV) to eliminate any potential for experimenter bias. The ASV method was conducted by taking 50 μL of whole blood mixed with 250 μL of hydro-chloric acid solution (0.34 M) and then applying the final mixture to the lead sensor strip and inserted into the ESA LeadCare II Blood Lead Analyzer system to determine BLLs. After 3 minutes, the BLLs were reported from the instrument in μg/dL with a lower sensitivity cut off value of 3 μg/dL and a high sensitivity cut off value of 65 μg/dL (i.e., SEM±1.5 μg/dL sensitivity detection level).

Establishing Operation for Motivational Learning

At PND 55 a naive set of Control (n=6), Perinatal (n=6), Perinatal+Taurine (n=6) male and female rats were scheduled for dig training and subsequently the ASST. In order to ensure that the rats had the necessary motivation to search for and consume a reward the following procedures were implemented as in the original ASST paper of Birrell & Brown (2000) and the methods of Neuwirth et al. (2019a): 1) rats were given a highly preferred food reward that consisted of a half piece of Kellogg's® Froot Loops® cereal; and 2) were placed on an approved National Institute of Health (NIH) (2017) Guidelines for Diet Control in Behavioral Studies (see for example the website at http://oacu.od.nih.gov/ARAC/dietctrol.pdf. This NIH approved food restriction schedule served to ensure that rats were maintained at a healthy 80% of their ad libitum body weight. The food restriction consisted of providing four food pellets to male and three food pellets to female rats daily. This procedure served to create a steady metabolic state and an establishing operation of motivation to search for and consume a food reward, during both the training and test session components comprising the ASST. The weights for each rat were taken as a baseline value prior to being placed on food restriction and continually monitored by being weighed every Monday, Wednesday, and Friday until testing was completed.

Dig Training

Following the establishment of the necessary motivational level for learning, at PND 55 male and female rats were scheduled for dig training. Dig training consisted of a rat searching within an acrylic bowl (711.2 mm L×431.8 mm W×406.4 mm H) in order to retrieve a half of a Kellogg's® Froot Loops® cereal piece within an increasing amount of shredded paper (i.e., the digging medium). Training consisted of rats being shaped through a sequence of five forward-chained behaviors during a 2-min trial: 1) empty bowls were sprinkled with ground Kellogg's® Froot Loops® cereal dust and half a cereal piece was placed in the center of the bowl; 2) bowls were prepared as before, but 25% of the bowl was filled with shredded paper; 3) bowls were prepared as before, but 50% of the bowl was filled with shredded paper; 4) the bowls were then filled to 75% with shredded paper; and 5) the bowl was then 100% filled with shredded paper. Rats had to complete 10-trials successfully for each digging sequence before moving to the next sequence to meet the criteria for being adequately dig trained. All dig trainings were completed in a single training session.

Attention Set-Shift Test (ASST)

The ASST was implemented consistent with the procedures of Birrell & Brown (2000) (for review of ASST methodology see Tait et al., 2018) and Neuwirth et al. (2019a) using the Neuwirth™ ASST apparatus. Between PNDs 56-90 dig-trained rats were subjected to a 4-day test schedule that was necessary to provide a test break for the Perinatal rats (i.e., negative reinforcement) consistent with the procedures of Neuwirth et al. (2019a). Briefly, the rats were given a two-choice pair stimulus presentation in which the bowls were lightly covered with ground Kellogg's® Froot Loops® cereal dust to prevent the rat from identifying the food reward based on scent alone. The criterion for a rat to move from one ASST condition to another was to complete 6-consequetive trials without an error.

On Test Day 1, the rat was presented with a 1^{st} set of novel stimuli parings as a two-choice presentation procedure. Each two-choice presentation consisted of discriminating between a pair of novel odors to the bowls (i.e., 20 μL of aromatic oils) and/or a pair of novel tactile medium (i.e., digging materials) within the acrylic bowls (see Table 1). The rats were then tasked to associate which stimulus was paired with the food reward (i.e., relevant stimulus) in comparison to the other stimulus/stimuli that was not paired with a food reward (i.e., irrelevant stimulus/stimuli). This served as either a simple discrimination (SD) between 2-stimuli pairings of either two-odors (i.e., an odor discrimination [OD]) or two-digging materials (i.e., a digging medium discrimination [MD]) (Table 1).

On Test Day 2, rats had to generalize what they learned from the 1^{st} set of novel stimuli parings for the OD and MD trainings using a new 2^{nd} d set of novel stimuli pairings to make a SD. Then the rats frontoexecutive functions were further challenged by being tasked to make a complex dis-crimination (CD) (i.e., now the two-choice presentation of bowls consisted of a combination of two odors and two digging medium at once [4-stimuli pairings] (Table 1). Following the CD, the rats cognitive flexibility was now challenged to ignore the previously relevant stimuli that was associated with the food reward and shift its attention to the previously irrelevant stimuli that was now paired with the food reward; thus, constituting a complex discrimination reversal (CD-Rev) task (Table 1).

On Test Day 3, the CD-Rev stage was re-tested (i.e., a learning reacquisition probe) to re-establish behavioral momentum through the ASST due to the required test break between test days. After the CD-Rev stage, the rat was presented with a 3^{rd} set of novel stimuli pairings and it was tasked with following the same relevant stimulus dimension (i.e., odor or digging medium from the prior day) in solving another CD, which served as an intradimensional shift (ID) (i.e., odor-to-odor or medium-to-medium “in the same relevant stimulus dimension as the prior test day to generalize learning”). This was followed by an intradimensional reversal (ID-Rev) (Table 1).

On Test Day 4, the ID-Rev was re-tested again with a learning re-acquisition probe to ensure behavioral momentum. Then the rat was presented with a 4^{th} anew set of novel stimuli pairings and it was tasked with following the previously irrelevant stimulus dimension (i.e., if the rat previously was following an odor stimulus it would now have to shift to a digging material stimulus) serving as the extradimensional shift (ED). This was followed by an extradimensional reversal (ED-Rev) (Table 1).

TABLE 1
The odor exemplar pairing using in the attention set-shifting task.

| TRAINING | ODORS | | DIGGING MEDIUM | |
|---|---|---|---|---|
| Pairing 1 | O1-Cumin | O2-Paprika | M1- | M2- |
| | | | Shredded | Polystyrene |
| | | | Paper | |
| Pairing 2 | 03- | 04- | M3- | M4- |
| SD, CD | White | Texas Cedar | Small Beads | Small Gravel |
| CD-Rev | Thyme | Wood | | |
| Pairing 3 | O5- | O6- | M5- | M6- |
| CD- | Clove | Rosemary | Fine Wood | Large Wood |
| Reacquisition, | Buds | | Shavings | Shavings |
| ID, ID-ReV | | | | |
| Pairing 4 | O7- | O8- | M7- | M8- |
| ID- | Spearmint | Cinnamon | Dirt with | Mulch |
| Reacquisition, | | | Wood | |
| ED, ED-Rev | | | shavings | |

Abbreviations are defined as follows: Pairing 2 comprised the (SD)=Simple Discrimination, (CD)=Compound Discrimination, and the (CD-Rev)=Compound Discrimination Reversal stages; Pairing 3 comprised the (CD-Reacquisition)=Compound Discrimination Retention, (ID)=Intra-dimensional Shift, and the (ID-Rev)=Intra-dimensional Reversal stages; and Pairing 4 comprised the (ID-Reacquisition)=Intra-dimensional Shift Retention, (ED)=Extra-dimensional shift, and the (ED-Rev)=Extra-dimensional Shift Reversal stages (Consistent with the procedures of Neuwirth et al., 2019a).

Brain Extractions and Sub-Region Dissections

Immediately following the ASST, rats were deeply anesthetized using Isoflurane, then sacrificed, and their brains were extracted in cold physiological buffered saline (PBS) pH 7.4 in under 2-min. The rat whole brains were then transferred into a coronal sectioning steel brain matrix for 175-300 g rodents (Stoelting, Inc. Wood Dale, Ill.). The whole rat brains were then manually sectioned into 1 mm thin slices using two sterile single-edged razor blades, transferred into Petri dishes containing cold PBS, and the following brain sub-regions were then manually dissected and collected into 1.5 mL tubes using a dissection microscope: prelimbic (PrL), infralimbic (IL), orbital ventral frontal (OV), orbital ventro-lateral (OVL), and hippocampal (HP) areas, respectively. The collected brain regions were stored at −80° C. until ready for subsequent neurochemical assessments.

Neurotransmitter Profile and Ratio Assessment

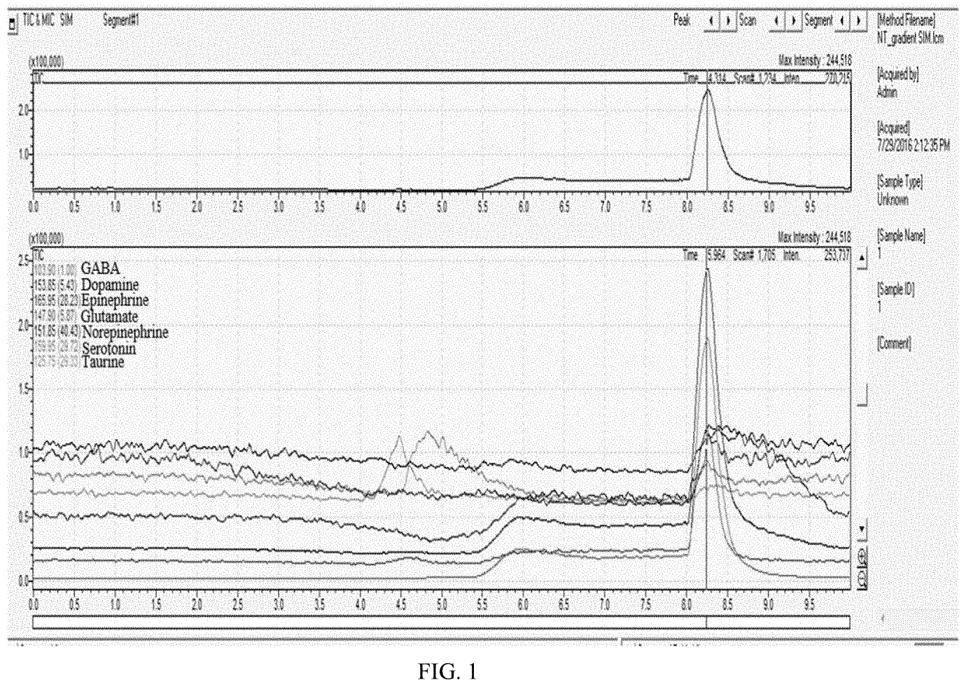

The brain sub-regions were then manually homogenized with sterile glass homogenizers (i.e., total volume 3 mL) using a 10 mg/100 μL (1:10) dilution of 100% acetonitrile (CHC₃N) (Sigma-Aldrich, St Louis, Mo.) as a miscible (i.e., fully dissolvable solution) with a dielectric constant to study the separation of chemicals by mass charge and polarity. Post homogenization, samples were sonicated for 30 sec with a pulse on:off time of 10 sec at an amplitude of 20%, then centrifuged at 14.8 RPM for 5-min at 4° C., and the supernatant collected and stored at −20° C. until ready for LC/MS. The supernatant was injected (i.e., 10 μL of pure brain sub-region sample) into a DC cell of a Shimadzu Liquid Chromatography/Mass Spectroscopy (LC/MS) 8030 (Shimadzu Scientific Instruments, Columbia, Md.) to assess the GABA and Taurine ratios to the following neuro-transmitters of interest: glutamate, norepinephrine, dopamine, serotonin, and epinephrine. Neurotransmitters were separated by High Performance Liquid Chromatography (HPLC) using a C18 reverse phase column. An acetonitrile gradient (0-100% acetonitrile in 0.1% TFA containing HPLC water) was used to separate different neurotransmitters. The mass/charge (m/z) values of neurotransmitters were monitored and peak heights were obtained to compare the amount of neurotransmitters within- and between-samples. The elution was performed with a flow rate of 0.2 mL/min and the neurotransmitters that were eluted from the column were detected in the positive ion mode. The spray voltage was kept at 5 kV and the capillary temperature was set at 250° C. and the sheath gas (nitrogen) was set at 60 units. Standards for LC/MS were made at a concentration of 1 mg/1 mL 100% acetonitrile from TLC grade (97-99.99%) chemicals from Sig-ma-Aldrich (St. Louis, Mo.) for the following neurotransmitters: γ-aminobutyric acid C₄H₉NO₂ (103.4 g/mol), Dopamine hydrochloride (HO)₂C₆H₃CH₂CH₂NH₂.HCL (153.85 g/mol), (−)-Epinephrine C₉H₁₃NO₃ (165.95 g/mol), D-glutamic acid C₅H₉NO₄ (147.90 g/mol), (−)-Norepinephrine C₈H₁₁NO₃ (151.85 g/mol), Serotonin hydrochloride C₁₀H₁₂N₂O.HCL (159.95 g/mol), and Taurine C₂H₇NO₃S₁ (125.75 g/mol) (FIG. 1). Referring now to FIG. 1, FIG. 1 illustrates the LC/MS detection profiles of the Sigma-Aldrich (St. Louis, Mo.) standards for the following neurotransmitters: GABA (103.4 g/mol), Dopamine (153.85 g/mol), Epinephrine (165.95 g/mol), Glutamate (147.90 g/mol), Norepinephrine (151.85 g/mol), Serotonin (159.95 g/mol), and Taurine (125.75 g/mol). Standards were made at a concentration of 1 mg/mL 100% acetonitrile.

Data Analyses

Data were recorded in real-time and analyzed using the Anymaze® video tracking software (Stoelting Co., Wood Dale, Ill.) transmitted via a ceiling mounted Logitech C310 Hi-speed USB 2.0 web camera (High-definition video with 1,280×720 pixels and 5 MP photo quality). The web camera was relayed to a standard Dell D16M Inspiron 3847 Desktop computer equipped with Windows 10 64-bit operating systems, 8 GB Dual Channel DDR3 1,600 MHZ (4 GB×2), 1 TB 7,200 PRM Hard Drive, and a 4^{th} Generation Intel® Core™ 3-4170 Processor (3 M Cache, 3.70 GHz), and displayed through a Dell 20″ E2016H monitor with an optimal resolution of 1,600×900 pixels at 60 Hz. Data were recorded as digital videos that were analyzed using AnyMaze® software. Animal tracking was based on contrast relative to the background. Different zones were labeled and indicated on the monitor. Three tracking points were specified by one on the rat's head, center of its body, and the last on its tail. An excel spread-sheet was generated containing all the parameters specified. The dependent variables of interest were the number of trials-to-criterion (TTC) and the number of errors-to-criterion (ETC). Additionally, data were analyzed using a cumulative record to observe the correct and error response differences in the rate-of-learning during each test condition of the ASST.

Data for the LC/MS samples were analyzed by taking the average intensity values of the neurotransmitter value (i.e., all values within +1 and −1), then divided all values by GABA to find the GABA:Neurotransmitter ratio. The same procedure was done for Taurine, by taking the average intensity value of the neurotransmitter and then dividing all values by Taurine to find the Taurine:Neurotransmitter ratio. A Microsoft Excel spreadsheet was generated containing all the respective GABA:Neurotransmitter and Taurine Neurotransmitter ratios specified.

Statistical Analyses

All behavioral data were collated in Microsoft Excel and later analyzed in IBM SPSS V. 24 (IBM, Inc. Armonk, N.Y.). For the ASST tests, an ANOVA was conducted using the ASST Test Condition as the within-subjects factors and ASST Test Condition and Treatment as the between-subjects factors for the dependent variables of TTC and ETC. For the LC/MS data, an ANOVA with Treatment and Brain Region as fixed-factors was used to evaluate the dependent variables of the GABA:Neurotransmitter and Taurine:NeurotransmitterRatios. The criteria for significance was set at α=0.05% with a 95% confidence interval with the data presented as the mean SEM. Significant differences were determined by an equal Tukey's HSD post hoc multiple comparisons tests along with a partial Eta-square ηₚ² for determining pairwise comparisons and effect sizes where applicable.

Results

The BLL data showed that Perinatal rats exhibited a range between 5.3-15 μg/dL at PND 22, with no significant differences as a function of taurine treatment. Between PNDs 56-90 after the rats had completed the ASST, their final blood draw reported BLLs below the ≤3 μg/dL detectable limit. This suggests that the Pb²⁺-exposure that was circulating throughout their cardiovascular system throughout development had been absorbed by bodily tissues and/or eliminated from the system after having already disrupted neurodevelopmental processes that would later contribute to frontoexecutive dysfunctions.

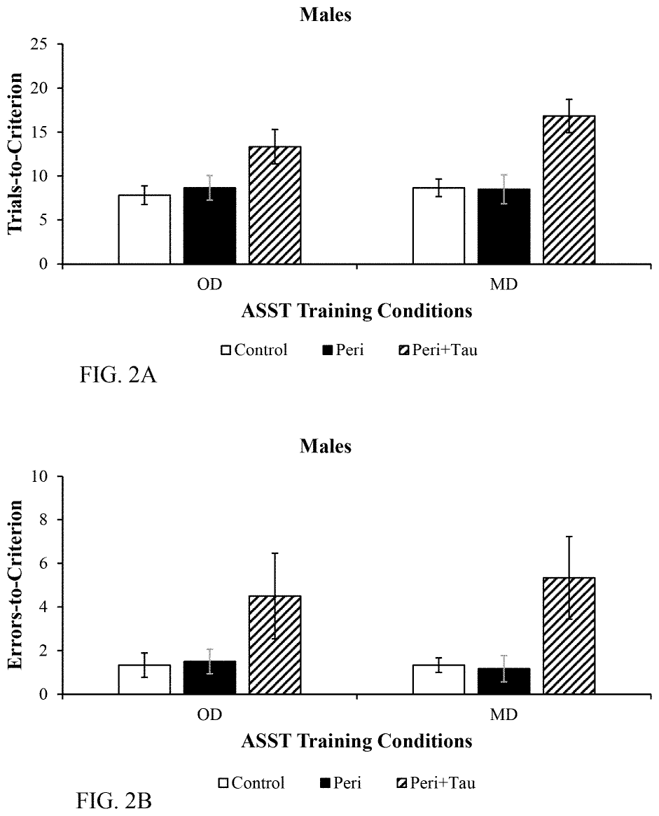

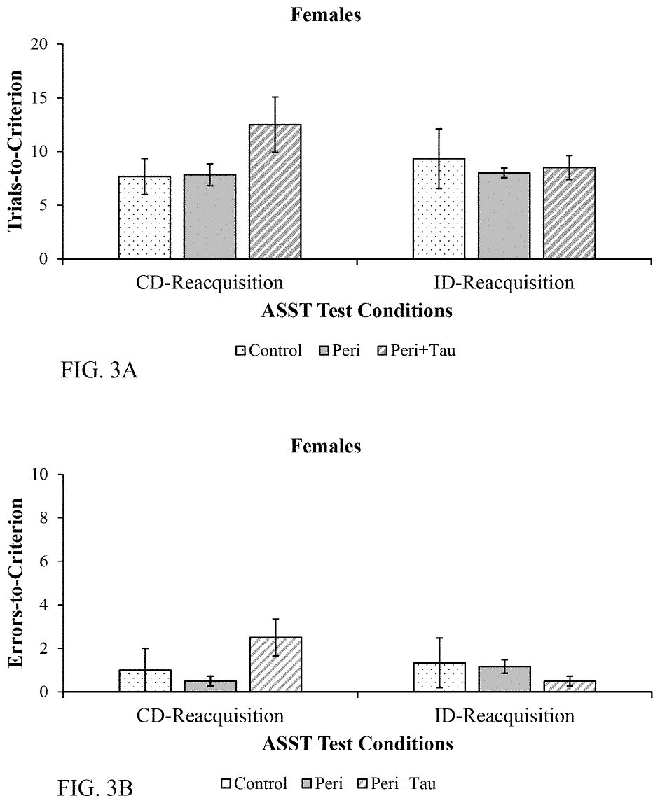

Prior to the ASST, rats were trained to dig through a medium to associate a reward through both odor (OD) and digging medium (MD) discriminations to examine their learning differences measured by the TTC and ETC. Control and Perinatal male rats showed no differences in learning the OD or MD for both TTC and ETC (FIG. 2A & FIG. 2B). However, Perinatal+Taurine male rats had significant difficulty in learning to make the OD and MD with Treatment effects for the TTC F₍₂₎=4.817, p<0.01^{##}, =ηₚ²=0.243 and the ETC F₍₂₎=6.023, p<0.01^{##}, (ηₚ²)=0.286 when compared to Control and Perinatal male rats (FIG. 2A & FIG. 2B). The data suggest that taurine co-treatment with developmental Pb²⁺-exposure may have induced a learning delay in these rats, but they were still capable of completing the ASST training. In contrast, Control, Perinatal, and Perinatal+Taurine female rats showed no differences in their OD and MD learning for the TTC or the ETC (FIG. 3A & FIG. 3B). Taken together, these data suggest sex-based differences in learning as a function of developmental Pb²⁺-exposure and taurine co-treatment.

Referring now to FIGS. 2A and 2B, FIG. 2A and FIG. 2B illustrate the differences in male rats' ability to learn odor (OD) and digging medium (MD) simple discriminations. The TTC (FIG. 2A) and the ETC (FIG. 2B) show that Control and Perinatal male rats learned at comparable rates. However, taurine co-treatment caused learning delays when compared to both Control and Perinatal male rats (p<0.01^{##}), respectively.

Referring now to FIGS. 3A and 3B, FIG. 3A and FIG. 3B illustrate the differences in female rats' ability to learn odor (OD) and digging medium (MD) simple discriminations. The TTC (FIG. 3A) and the ETC (FIG. 3B) show that Control and Perinatal female rats learned at comparable rates.

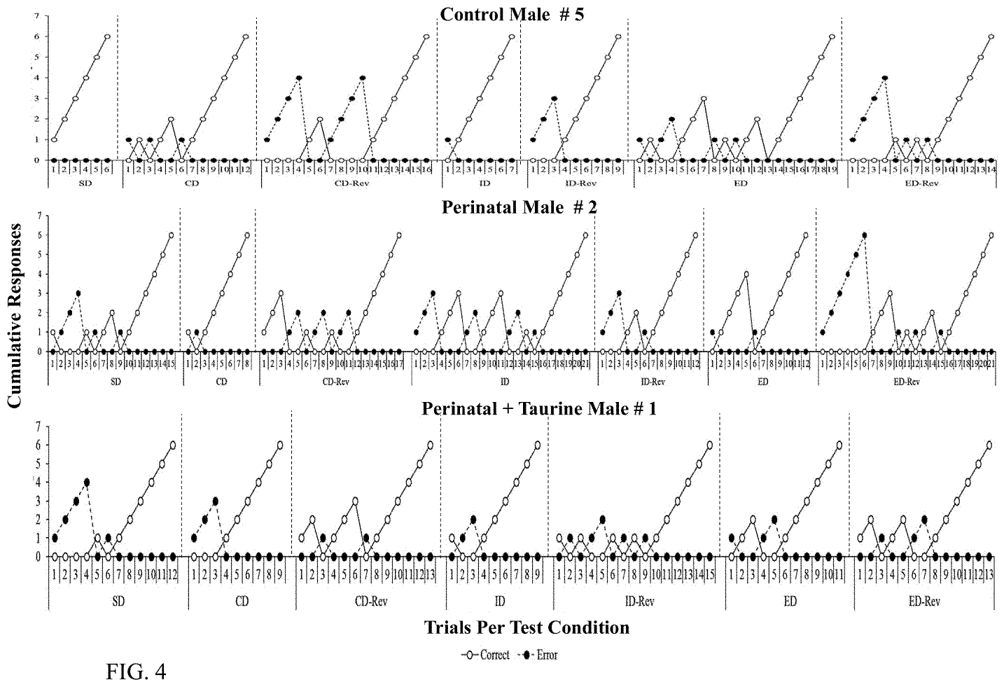

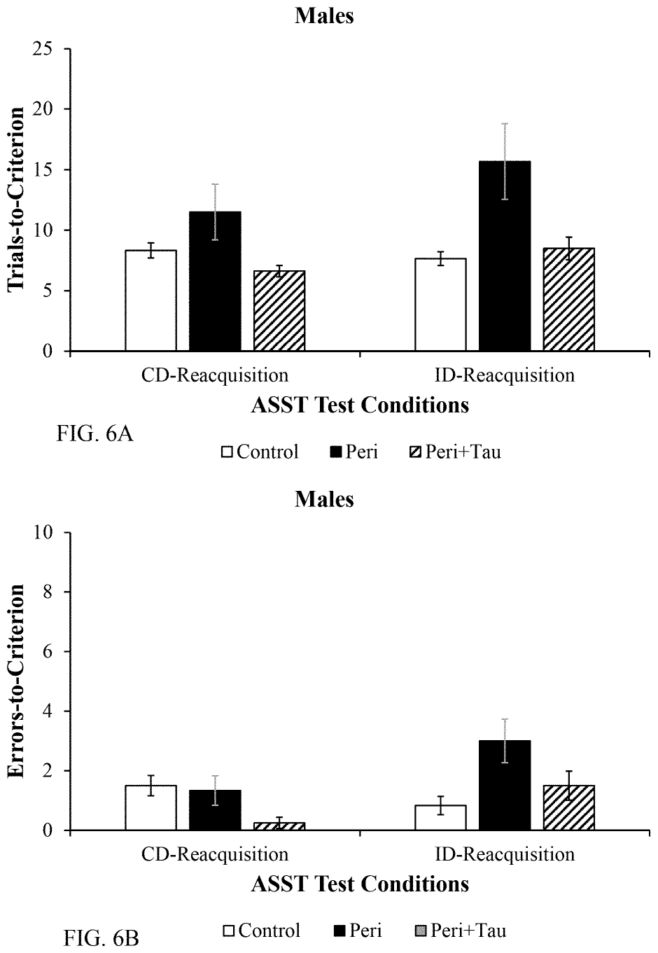

At PND 22 the perinatal Pb²⁺-exposed rats were removed from the neurotoxicant exposure for the remainder of the study. The effects of this developmental Pb²⁺-exposure caused persistent frontoexecutive dysfunctions in a sex-dependent manner that was observed within the ASST. In order to examine the individual rats' ASST performance differences, a representative sample from each gender and treatment condition were randomly selected. The individual rats' performance data regarding their correct and error response differences during their rate-of-learning cumulative records across the test conditions of the ASST, showed that developmental Pb²⁺-exposure caused significant frontoexecutive impairments and delays in and accuracy of correct responses for both male (FIG. 4) and female rats (FIG. 6). Female rats required a greater number of trials to complete the ASST with the most difficulty observed in the ED-Rev test condition. The data suggest that female rats were more negatively affected by Pb²⁺-exposure than males as evidenced by increased trials required to complete the ED and ED-Rev test conditions of the ASST. Interestingly, these individual within-subject behavioral performances showed significant improvements in response to taurine co-treatment; thereby, mitigating Pb²⁺-exposure in reducing these frontoexecutive dysfunctions.

Referring now to FIG. 4, FIG. 4 illustrates the rate-of-learning cumulative records for a single representative male rat from the Control (upper panel), Perinatal (middle panel), and Perinatal+Taurine (lower panel) treatment groups. The data show the 7-test conditions of the ASST (separated within each panel by the vertical dashed phase-lines) along the x-axis and the number of cumulative responses on the y-axis, with the correct responses (open circles with solid lines) and the error responses (black circles with dashed lines) are depicted as the rats' rate-of-learning. Control male rats make fewer errors throughout the 7-test conditions of the ASST, when com-pared to the Perinatal male rats. Control male rats' make sequential errors during the CD-Rev, ID, ID-Rev, ED, and ED-Rev ASST stages. In contrast, the Perinatal male rat makes sequential errors in the SD, CD-Rev, ID, ID-Rev, and ED-Rev ASST stages. Interestingly, the Perinatal+Taurine male rat exhibited a quicker rate-of-learning with less sequential errors during the SD, CD, ID, ID-Rev, ED, and ED-Rev ASST stages. The data suggest that developmental Pb²⁺-exposure induces lasting frontoexecutive dysfunctions in the mature rats' rate-of-learning behavioral profile, which improved by the co-treatment of Taurine 0.05% developmentally during Pb²⁺-exposure.

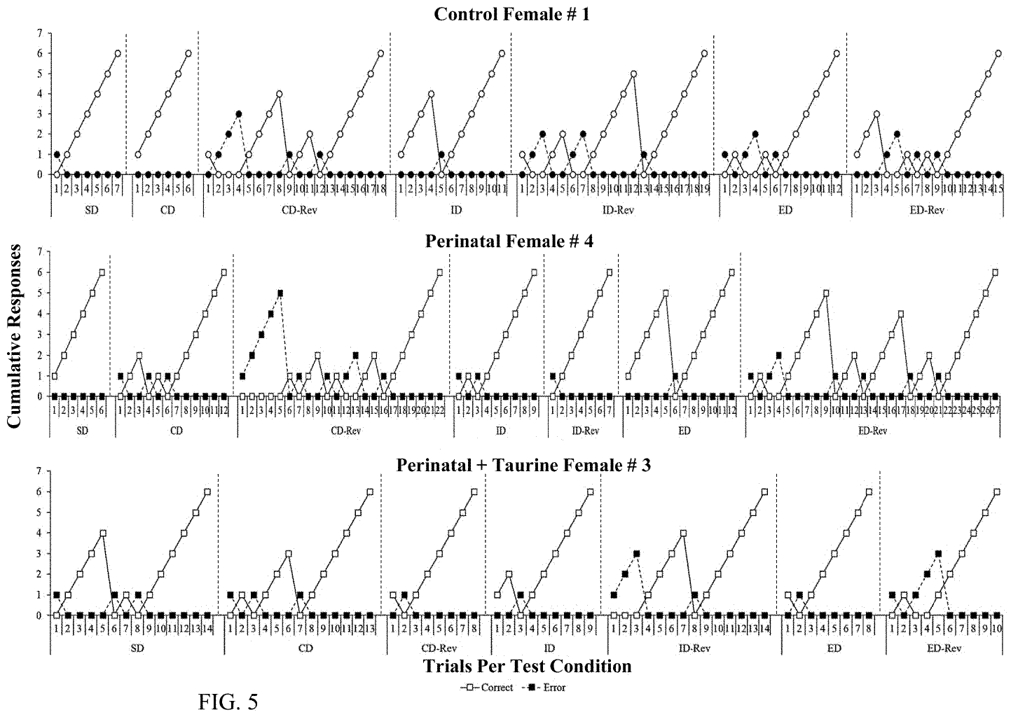

Referring now to FIG. 5, FIG. 5 Illustrates the rate-of-learning cumulative records for a single representative female rat from the Control (upper panel), Perinatal (middle panel), and Perinatal+Taurine (lower panel) treatment groups. The data show the 7-test conditions of the ASST (separated within each panel by a vertical dashed-line) along the x-axis and the number of cumulative responses on the y-axis, with the correct responses (open squares with solid lines) and the error responses (black squares with dashed lines) are depicted as the rats' rate-of-learning. Control female rats make fewer errors throughout the 7-test conditions of the ASST, when compared to the Perinatal female rats. Control female rats' make sequential errors during the CD-Rev, ID-Rev, ED, and ED-Rev ASST stages. In contrast, the Perinatal female rat makes sequential errors in the CD-Rev and ED-Rev ASST stages. Interestingly, the Perinatal+Taurine female rats exhibited a quicker rate-of-learning with less sequential errors during the ID-Rev and ED-Rev ASST stages. The data suggest that developmental Pb²⁺-exposure induces lasting frontoexecutive dysfunctions in the mature rats' rate-of-learning behavioral profile, which improved by the co-treatment of Taurine 0.05% developmentally during Pb²⁺-exposure with more sensitivity when compared to male rats.

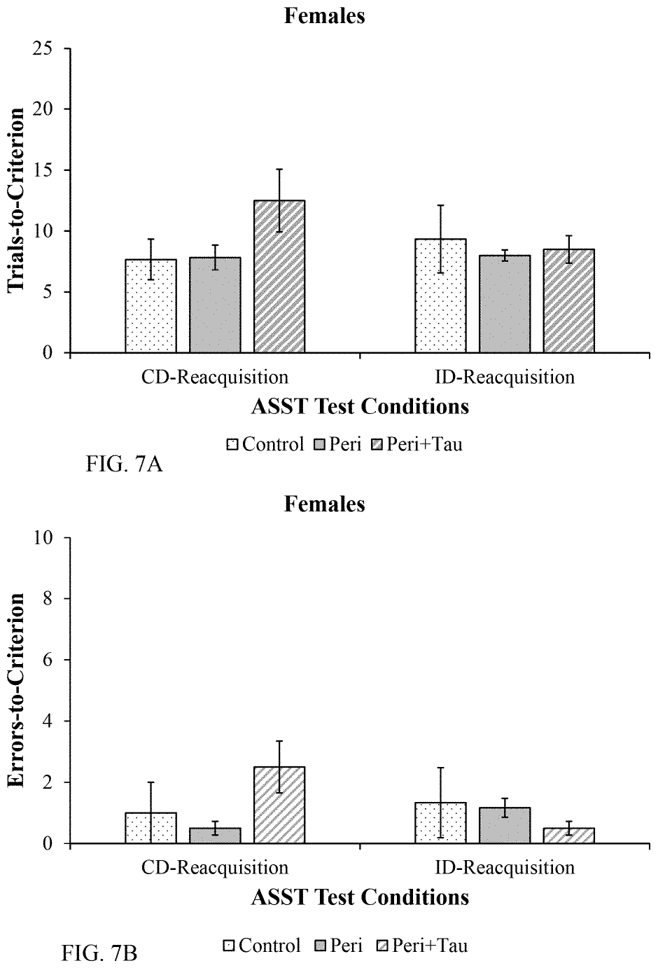

Developmental Pb²⁺-exposure caused deficits in the ASST re-acquisition learning performance that was recovered by taurine co-treatment. During the ASST, a test break procedure was implemented consistent with reports by Neuwirth et al. (2019a). As such, a re-acquisition learning probe was used for the CD and ID (i.e., CD-Reacquisition and ID-Reacquisition) to ensure the behavioral momentum to evaluate the rats' cognitive flexibility in shifting could be maintained. Perinatal male rats showed a significant increase in TTC for OD and MD as a Treatment effect F₍₂₎=7.405, p<0.001***, (ηₚ²)=1=0.331, when compared to Control male rats (FIG. 6A). Further, Perinatal+Taurine male rats showed a recovery from the TTC reacquisition learning impairment for both the OD (p<0.01^{##}) and MD (p<0.01^{##}) (FIG. 6A). Additionally, Perinatal male rats showed a significant decrease in ETC for OD and MD as a Treatment effect F₍₂₎=3.458, p<0.05*, (ηₚ²)=0.187, when compared to Control male rats, as well as, an ASST Stage×Treatment interaction F_{(6,2)}=4.031, p<0.05*, (ηₚ²)=0.212 (FIG. 6B). Consistent with the TTC reacquisition learning data, Perinatal+Taurine male rats showed a fewer ETC errors in both the OD (p<0.05^{#}) and MD (p<0.05^{#}), corroborating the finding that taurine co-treatment improved reacquisition learning deficits (FIG. 6B). In contrast, Control, Perinatal, and Perinatal+Taurine female rats showed no differences in both TTC and ETC OD and MD performances, respectfully (FIG. 7A & 7B). Taken together, the data suggest that developmental Pb²⁺-exposure caused reacquisition learning deficits in a sex-dependent manner with males being most affected, and these impairments were recovered in males by taurine co-treatment.

Referring now to FIGS. 6A and 6B, FIGS. 6A and 6B illustrate the male rat reacquisition learning data between test days to ensure their behavioral momentum when advancing to the next ASST condition. The reacquisition learning data show the TTC (FIG. 6A) and ETC (FIG. 6B) performances, respectively. Perinatal male rats showed a significant increase in the TTC required to complete the CD-Reacquisition and ID-Reacquisition (p<0.001***), as well as the ETC in the ID-Reacquisition (p<0.05*), when compared to Control male rats. Interestingly, co-treatment with taurine recovered reacquisition learning performance deficits to rates comparable to Control male rats for both the CD-Reacquisition and ID-Reacquisition in the TTC (p<0.001^{###}) and ETC (p<0.05^{#}). Thus, the data suggest that co-treatment with taurine improved ASST reacquisition learning performance in Perinatal Pb²⁺-exposed rats.

Referring now to FIGS. 7A and 7B, FIGS. 7A and 7B illustrates the female rat reacquisition learning data between test days to ensure their behavioral momentum when advancing to the next ASST condition. The reacquisition data show the TTC (FIG. 7A) and ETC (FIG. 7B) performances, respectively. There were no differences observed in female rats TTC and ETC as a function of treatment for either the CD-Reacquisition or ID-Reacquisition. Thus, unlike male rats, developmental Pb²⁺-exposure did not impair female rats' ASST reacquisition learning performance.

Developmental Pb²⁺-exposure caused frontoexecutive dysfunction impeding rats' ASST performance in a sex-dependent manner that was recovered by the co-treatment of taurine.

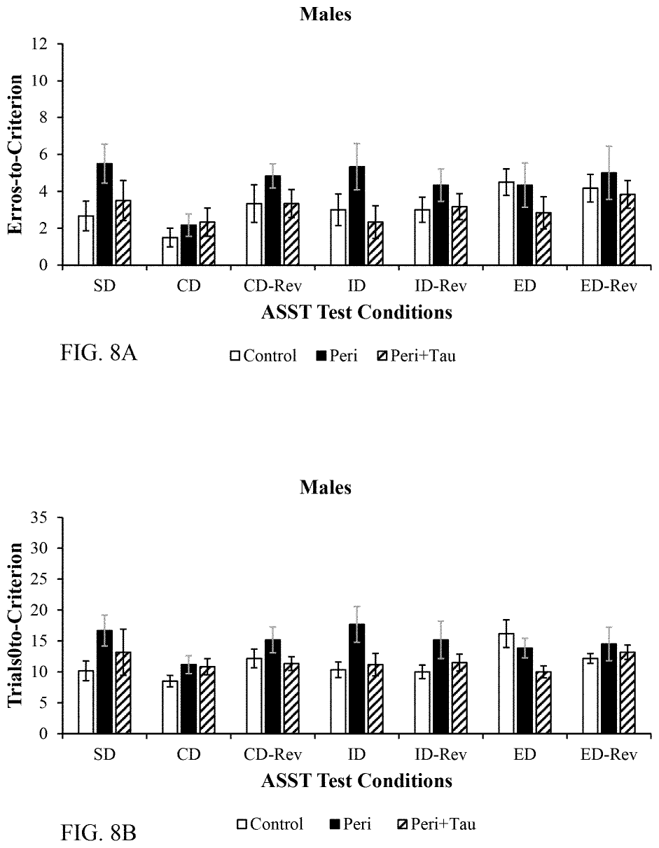

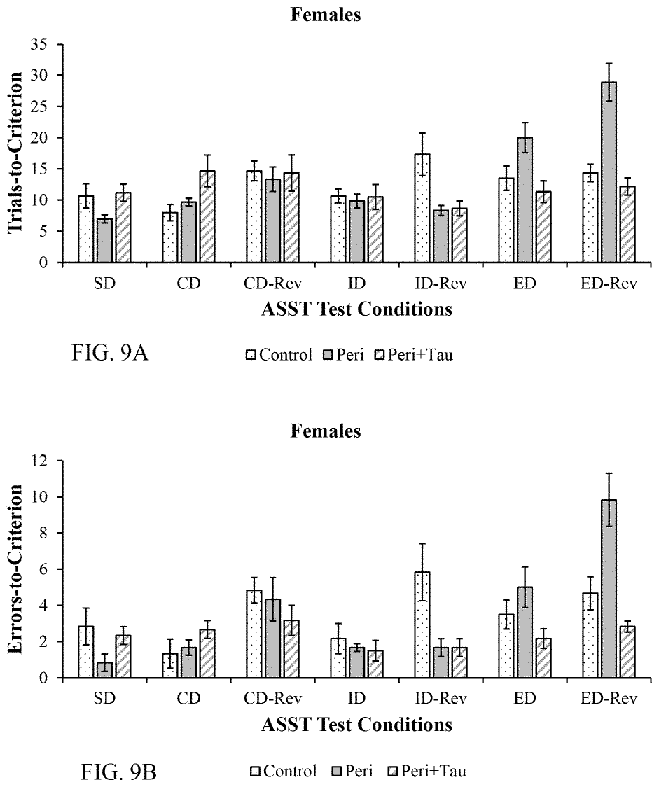

The ASST is a very sensitive behavioral test for frontoexecutive (dys)functions in rats. Consistent with reports by Neuwirth et al. (2019a), Perinatal male rats showed a significant Treatment effect in both TTC F₍₂₎=7.260, p<0.01**, (ηₚ²)=0.121 and ETC F₍₂₎=5.648, p<0.01**, (ηₚ²)=0.097 performances (FIG. 8A & FIG. 8B). Perinatal male rats showed the most difficulty at the SD, ID, and ID-Rev (p<0.01**) for the TTC and the SD and ID (p<0.01**) for the ETC test conditions. Moreover, taurine co-treatment recovered these deficits to performance levels comparative to Control males with the most recovery observed in the TTC during the CD-Rev, ID, and ED (p<0.01^{##}) and in the ETC during CD-Rev and ID (p<0.01^{##}) (FIGS. 8A & 8B). In contrast, the Perinatal females showed a significant effect of ASST Stage F₍₆₎=7.107, p<0.001***, (ηₚ²)=0.289, but no significant Treatment effects for TTC and a significant ASST Stage×Treatment interaction for the TTC F_{(6,2)}=8.277, p<0.001**, (ηₚ²)=0.486. Additionally, there was a significant effect of ASST Stage F₍₆₎=7.030, p<0.001***, (ηₚ²)=0.287, Treatment F₍₂₎=5.638, p<0.01**, (ηₚ²)=0.097, and a significant ASST Stage×Treatment interaction F_{(6,2)}=5.846, p<0.001***, (ηₚ²)=0.401 for ETC (FIGS. 9A & 9B). Perinatal female rats showed increased performance at the ID-Rev (p<0.05*), and increased difficult at the ED (p<0.01**) and ED-Rev (p<0.001***) for the TTC. In contrast, Perinatal female rats showed increased performance in the ID-Rev (p<0.05*) and increased difficulty at the ED-Rev (p<0.001***) for the ETC test conditions. Moreover, taurine co-treatment recovered these deficits to performance levels comparative to Control males with the most recovery observed in the TTC during the ED and ED-Rev (p<0.05^{#}) and in the ETC during ED and ED-Rev (p<0.05^{#}) (FIG. 9A & 9B). Taken together, the data suggest that the ASST is very sensitive in detecting the frontoexecutive dysfunctions caused by developmental Pb²⁺-exposure and the recovery of these cognitive behavioral performance deficits by the co-treatment of taurine in a sex-dependent manner.

Referring now to FIGS. 8A and 8B, FIGS. 8A and 8B illustrate the male rats ASST performance for TTC (FIG. 8A) and ETC (FIG. 8B) performance, respectively. Perinatal male rats showed a significant increase in the TTC required to complete the SD, ID, and ID-Rev (p<0.01**), as well as the ETC in the SD and ID (p<0.01***), when compared to Control male rats. Interestingly, co-treatment with taurine recovered ASST performance to rates comparable to Control male rats for both the CD-Rev, ID, ID-Rev, and ED in the TTC (p<0.01^{##}) and in the ETC (p<0.01^{##}). Thus, the data suggest that co-treatment with taurine recovered ASST frontoexecutive functions in Perinatal Pb²⁺-exposed rats.

Referring now to FIGS. 9A and 9B, FIGS. 9A and 9B illustrate the female rats ASST performance for TTC (FIG. 9A) and ETC (FIG. 9B) performance, respectively. Perinatal female rats showed a significant decrease in the TTC required to ID-Rev (p<0.05*) and a significant increased to complete the ED (p<0.01**), and ED-Rev (p<0.001***), as well as significant decrease in the ETC in the ID-Rev (p<0.05*) and a significant increase to complete the ED-Rev (p<0.001***), when compared to Control female rats. Interestingly, co-treatment with taurine recovered ASST performance to rates comparable to Control female rats for both the ED and ED-Rev in the TTC (p<0.05^{#}) and in the ED (p<0.05^{#}), and ED-Rev in the ETC (p<0.05^{#}). Thus, the data suggest that co-treatment with taurine recovered ASST frontoexecutive functions in Perinatal Pb²⁺-exposed rats.

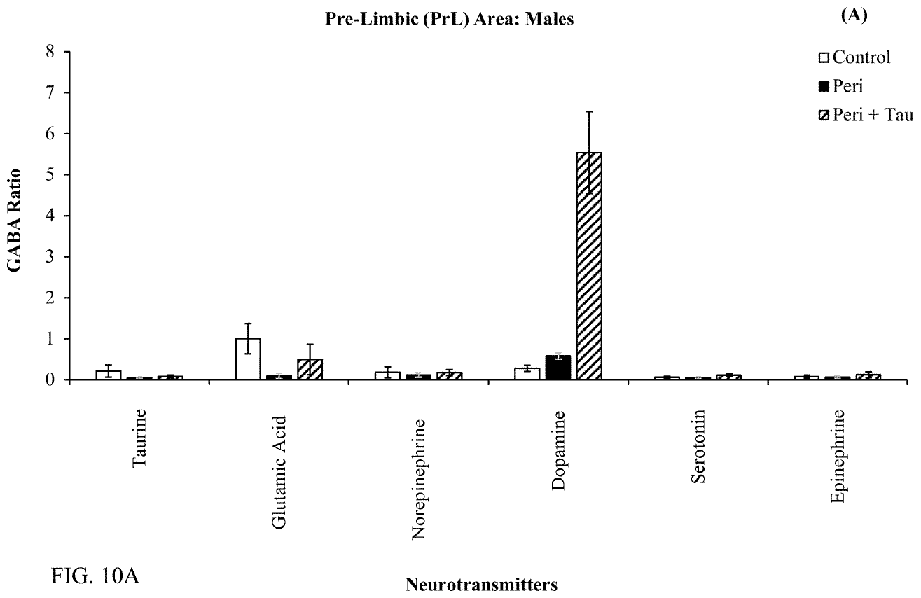

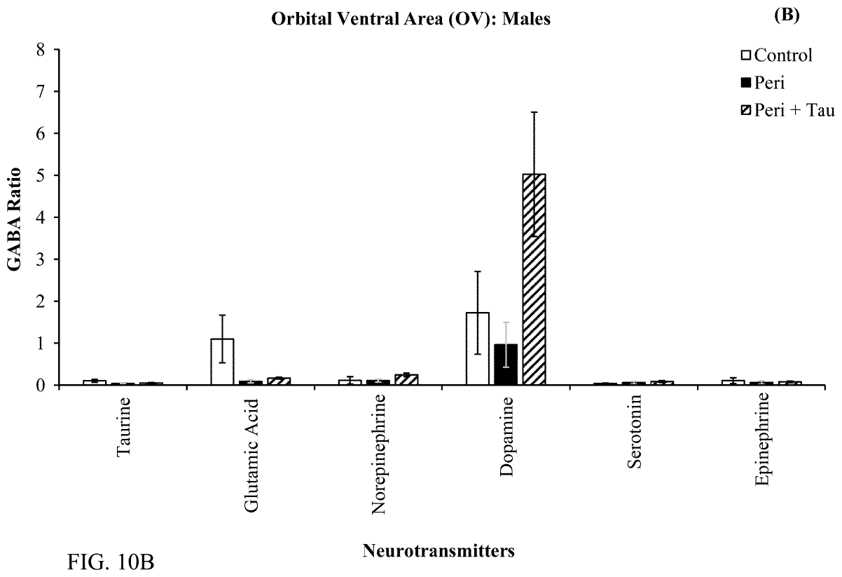

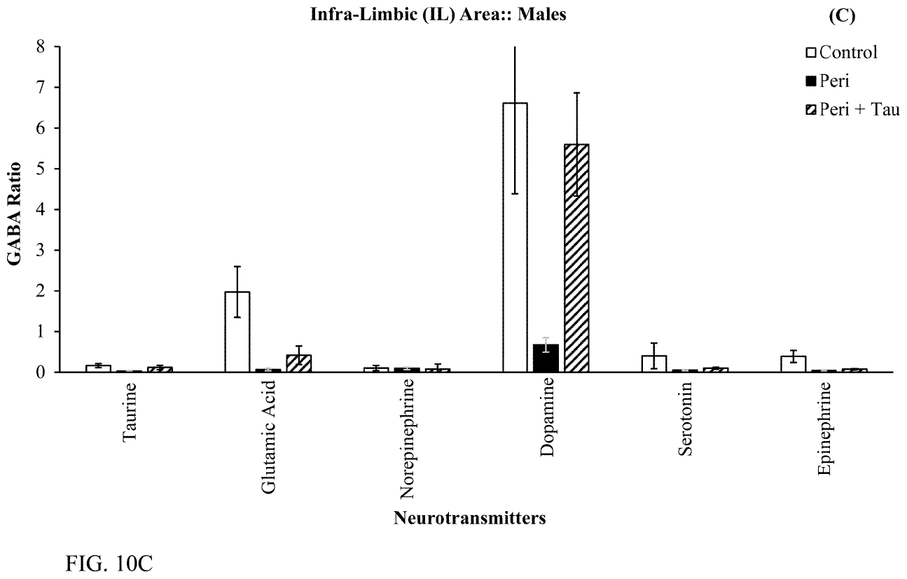

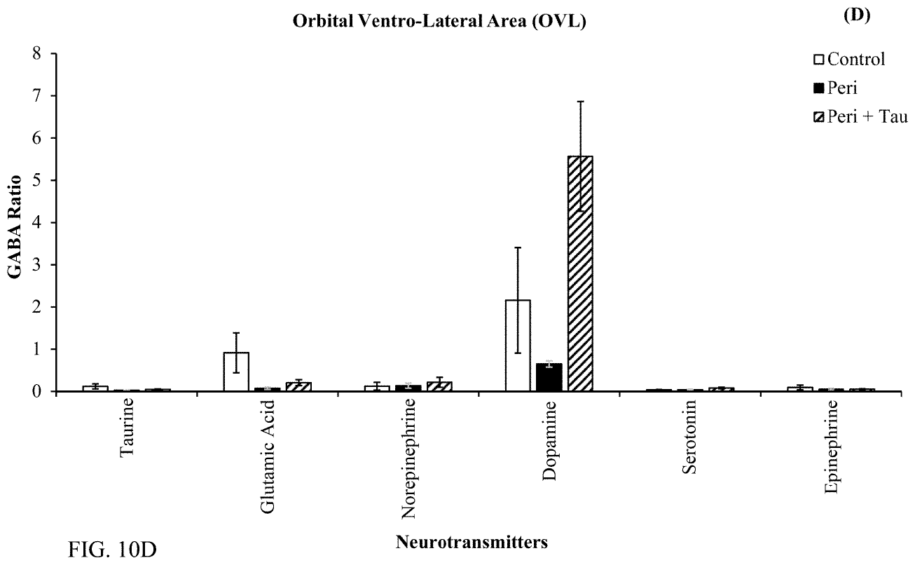

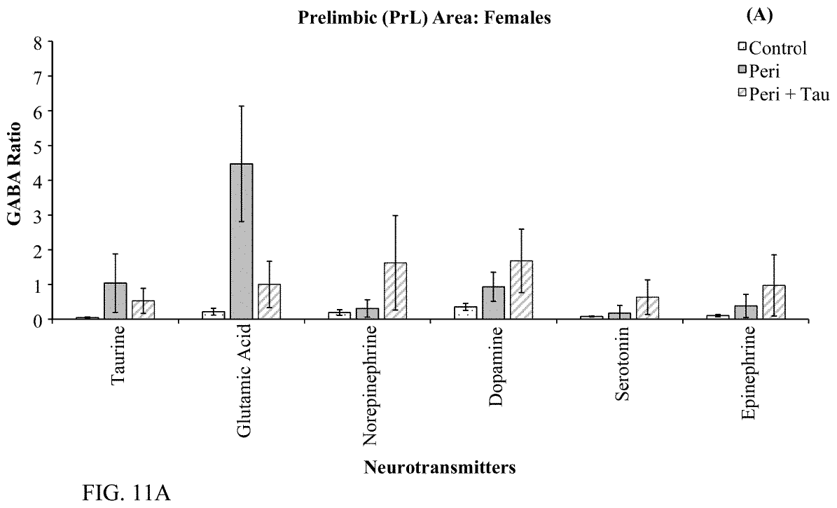

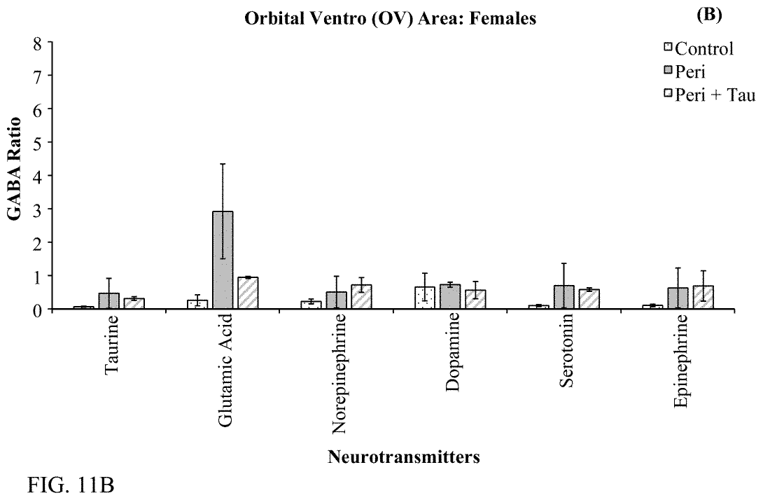

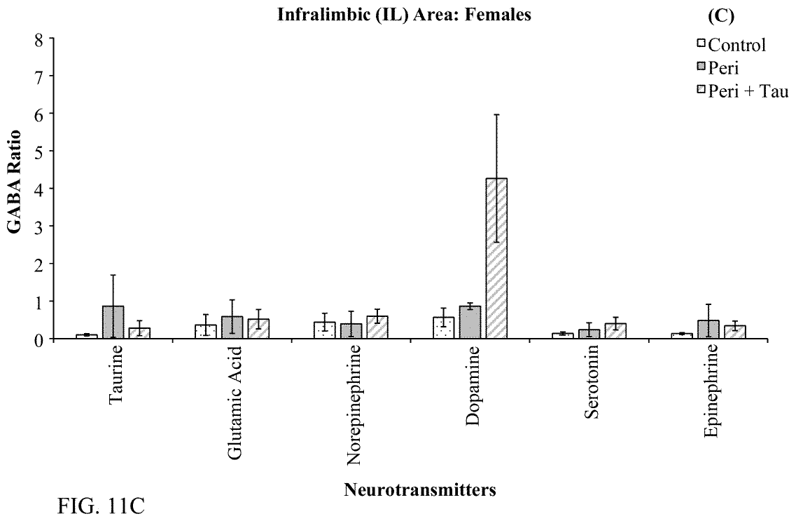

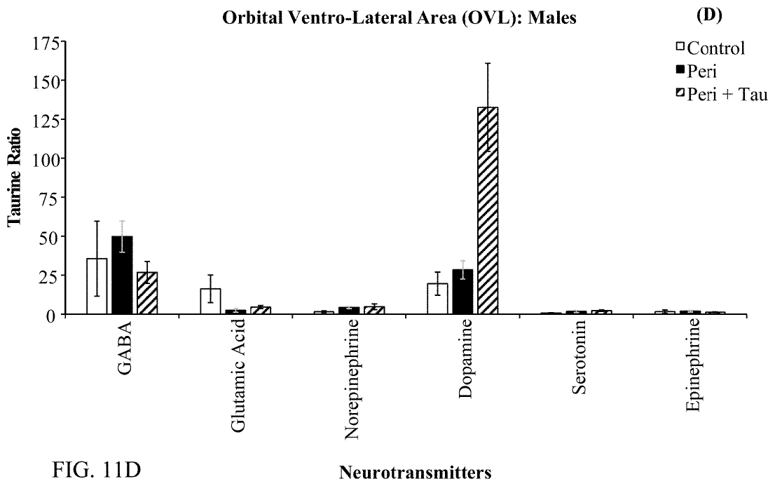

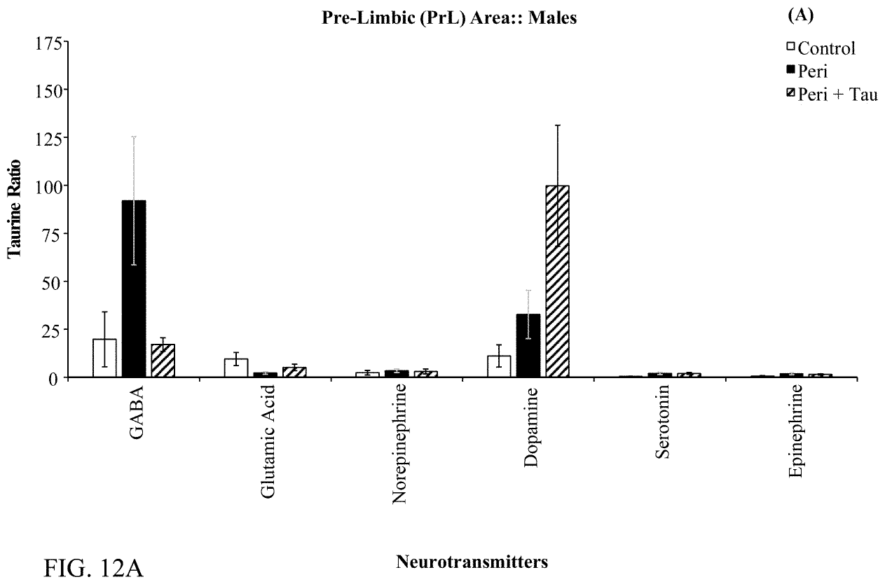

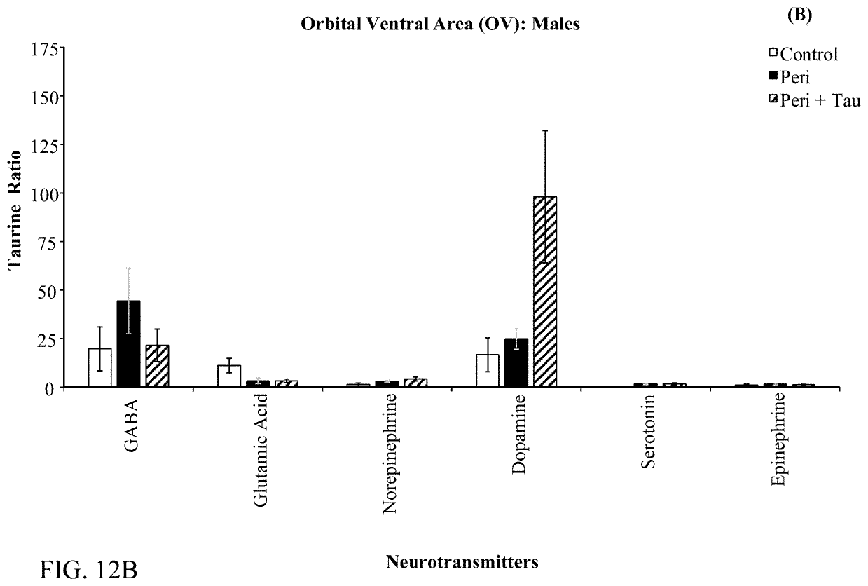

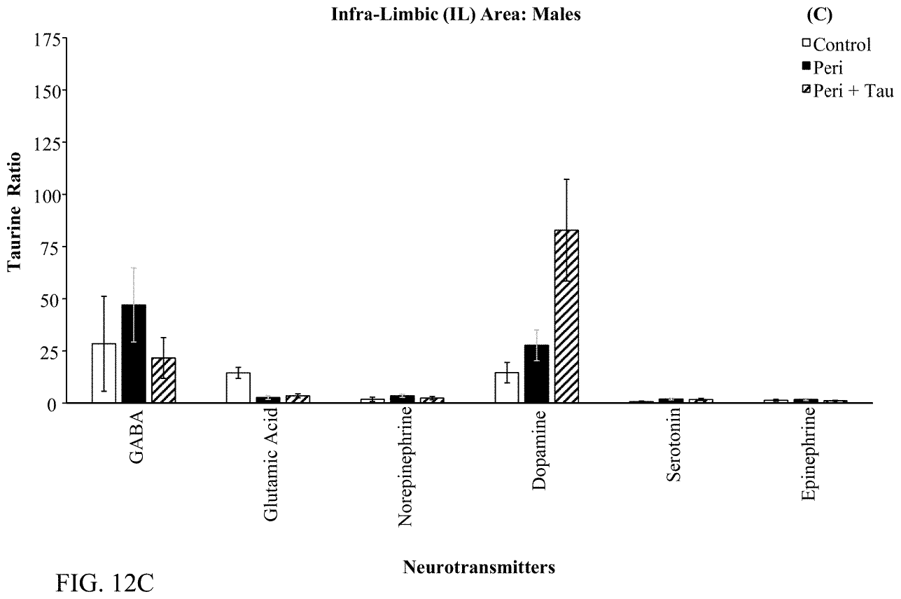

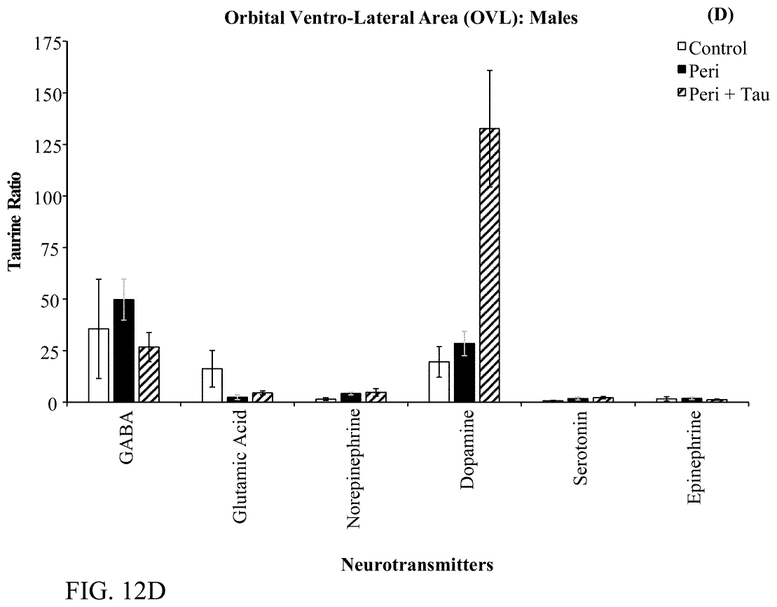

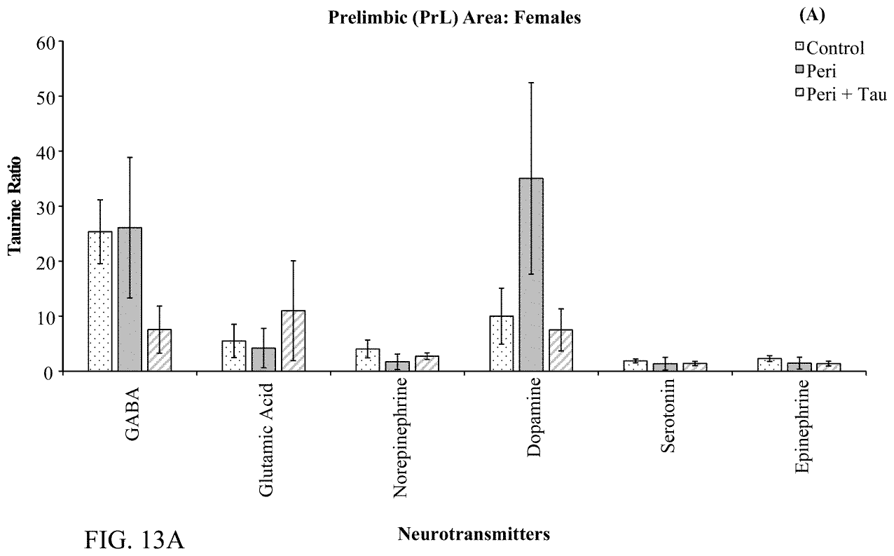

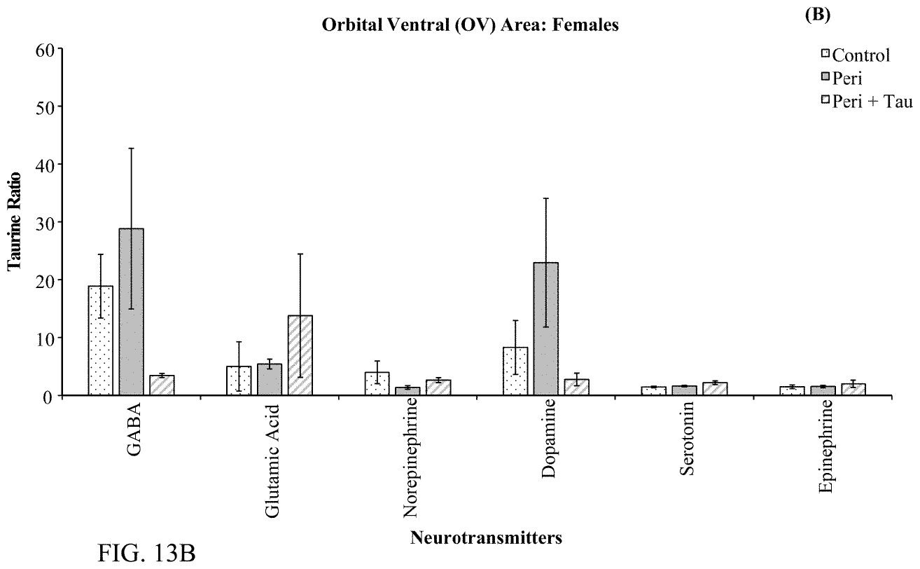

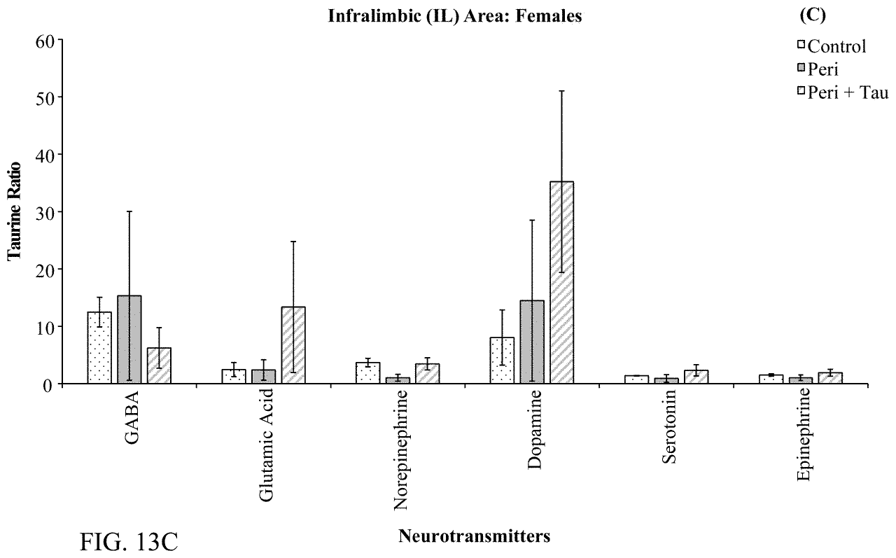

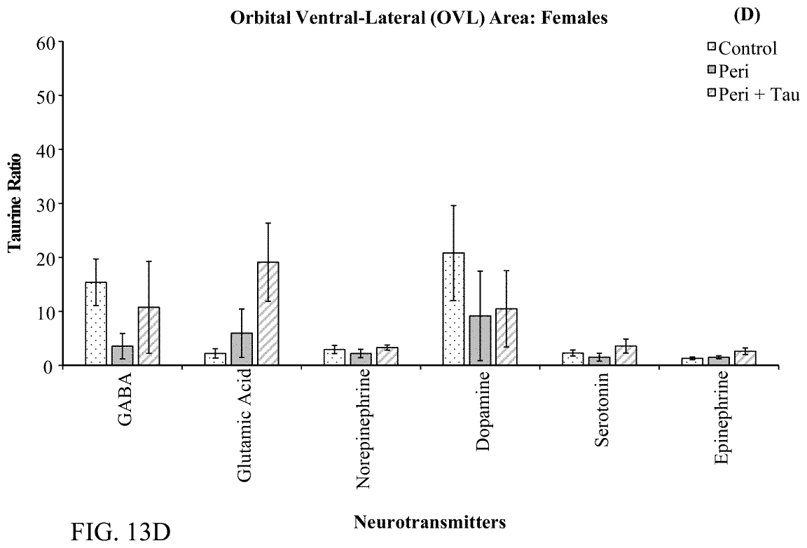

Developmental Pb²⁺-exposure caused altered neurochemical profiles in brain regions that serve to regulate frontoexecutive functions and are recovered by taurine co-treatment. To corroborate the frontoexecutive functions observed at the behavioral level, LC/MS analyses were conducted from the brain tissues of rats that completed the ASST. The brain regions examined were selected since they are known to play a critical role in frontoexecutive control and learning and memory. The data for the male rats GABA:Neurotransmitter ration an effect of Treatment was observed for GABA:Taurine F₍₂₎=4.044, p<0.01**, (ηₚ²)=0.142, GABA:Glutamic Acid F₍₂₎=13.456, p<0.001***, (ηₚ²)=0.355, GABA:Dopamine F₍₂₎=17.880, p<0.001***, (ηₚ²)=0.422 (FIG. 10). In addition, the GABA:Dopamine ratio showed a significant effect of Brain Region for the IL F₍₄₎=3.741, p<0.01**, (ηₚ²)=0.234, with a Treatment×Brain Region interaction F_{(2,4)}=2.796, p<0.01**, (ηₚ²)=0.313 (FIG. 10 & FIG. 13). Additionally, the data for male rats Taurine:Neuro-transmitter ratio an effect of Treatment was observed for Taurine:GABA F₍₂₎=5.156, p<0.01**, (ηₚ²)=0.177, Taurine:Glutamic Acid F₍₂₎=9.701, p<0.001***, (ηₚ²)=0.288, Taurine:Dopamine F₍₂₎=23.600, p<0.001***, (ηₚ²)=0.496, Taurine:Serotonin F₍₂₎=4.419, p<0.01**, (ηₚ²)=0.155, and Taurine:Epinepherine F₍₂₎=8.305, p<0.001***, (ηₚ²)=0.257 (FIG. 12 & FIG. 13). For the Taurine:Neurotransmitter ratio, an effect of Brain Region for the HP was observed for GABA:Taurine F₍₄₎=4.512, p<0.001***, (ηₚ²)=0.273, Taurne:Serotonin F₍₄₎=4.115, p<0.01**, (ηₚ²)=0.255, and Taurine:Epinepherine F₍₄₎=9.710, p<0.001***, (ηₚ²)=0.447 (FIG. 13). In contrast, for female rats the GABA:Neurotransmitter ratio an effect of Treatment was observed for GABA:Taurine F₍₂₎=6.242, p<0.01**, (ηₚ²)=0.301, GABA:Glutamic Acid F₍₂₎=4.127, p<0.01**, (ηₚ²)=0.216, GABA:Norepinephrine F₍₂₎=5.089, p<0.01**, (ηₚ²)=0.260, GABA:Serotonin F₍₂₎=5.789, p<0.01**, (ηₚ²)=0.278, and GABA:Epinephrine F₍₂₎=4.597, p<0.01**, (ηₚ²)=0.235 (FIG. 11 & FIG. 13). In regards to the female rats Taurine:Neurotransmitter ratio, and effect of Treatment was observed for Tauine:Glutamic Acid F₍₂₎=4.560, p<0.01**, (ηₚ²)=0.239, while an effect of Brain Region was observed for the HP in Tauine:Norepinepherine F₍₄₎=2.814, p<0.05*, (ηₚ²)=0.327, TaurineSerotonin F₍₄₎=3.129, p<0.01**, (ηₚ²)=0.350, and Taurine:Epinepherine F₍₄₎=3.809, p<0.01**, (ηₚ²)=0.396 (FIG. 13).

Referring now to FIGS. 10A-10D, FIGS. 10A-10D illustrates the male rats LC/MS GABA:Neurotransmitter ratios in the pre-limbic (PrL) (FIG. 10A), the orbital ventral (OV) (FIG. 10B), infralimbic (IL) (FIG. 10C), and the orbital ventro-later (OVL) (FIG. 10D) areas of the prefrontal cortex that regulate frontoexecutive functions. The data reveal that in the IL, OV, and OVL Perinatal Pb²⁺-exposures reduce GABA:Dopamine ratios. Following taurine co-treatment, these GABA:Dopamine ratios are reversed back to levels comparable to or exceeding those of Control males. The data suggest that Pb²⁺-exposure negatively effects GABA:Dopamine frontoexecutive signaling at the neurochemical level, which could reduce motivational states at the behavioral level.

FIGS. 11A-D illustrates the female rats LC/MS GABA:Neurotransmitter ratios in the prelimbic (PrL) (FIG. 11A), the orbital ventral (OV) (FIG. 11B), infralimbic (IL) (FIG. 11C), and the orbital ventro-later (OVL) (FIG. 11D) areas of the prefrontal cortex that regulate frontoexecutive functions. The data reveal that in the PrL, OV, and OVL Perinatal Pb²⁺-exposures increases GABA:Glutamic Acid ratios. Following taurine co-treatment, these GABA:Glutamic Acid ratios are reversed back to levels comparable to or exceeding those of Control females. Additionally, in the IL an elevation in GABA:Dopamine was observed following taurine co-treatment. The data suggest that Pb²⁺-exposure negatively effects GABA:Glutamic Acid and GABA:Dopamine frontoexecutive signaling at the neurochemical level, which could reduce motivational states at the behavioral level.

FIGS. 12A-12D illustrates the male rats LC/MS Taurine:Neurotransmitter ratios in the pre-limbic (PrL) (FIG. 12A), the orbital ventral (OV) (FIG. 12B), infralimbic (IL) (FIG. 12C), and the orbital ventro-later (OVL) (FIG. 12D) areas of the prefrontal cortex that regulate frontoexecutive functions. The data reveal that in the PrL Perinatal Pb²⁺-exposures increase Taurine:GABA ratios. Following taurine co-treatment, these Taurine: GABA ratios are reversed back to levels comparable to or less than Control males. Additionally, taurine co-treatment elevated Taurine:Dopamine levels across all four brain regions. The data suggest that Pb²⁺-exposure alters Taurine:GABA and Taurine: Dopamine frontoexecutive signaling at the neurochemical level, which could re-duce motivational states at the behavioral level.

FIGS. 13A-13D illustrates the female rats LC/MS Taurine:Neurotransmitter ratios in the prelimbic (PrL) (FIG. 13A), the orbital ventral (OV) (FIG. 13B), infralimbic (IL) (FIG. 13C), and the orbital ventro-later (OVL) (FIG. 13D) areas of the prefrontal cortex that regulate frontoexecutive functions. The data reveal that in the PrL and OV Perinatal Pb²⁺-exposures increase Taurine:Dopamine ratios. Following taurine co-treatment, these Taurine:Dopamine ratios are reversed back to levels comparable to or less than Control females. The data suggest that Pb²⁺-exposure alters Taurine:Dopamine frontoexecutive signaling at the neurochemical level, which could reduce motivational states at the behavioral level.

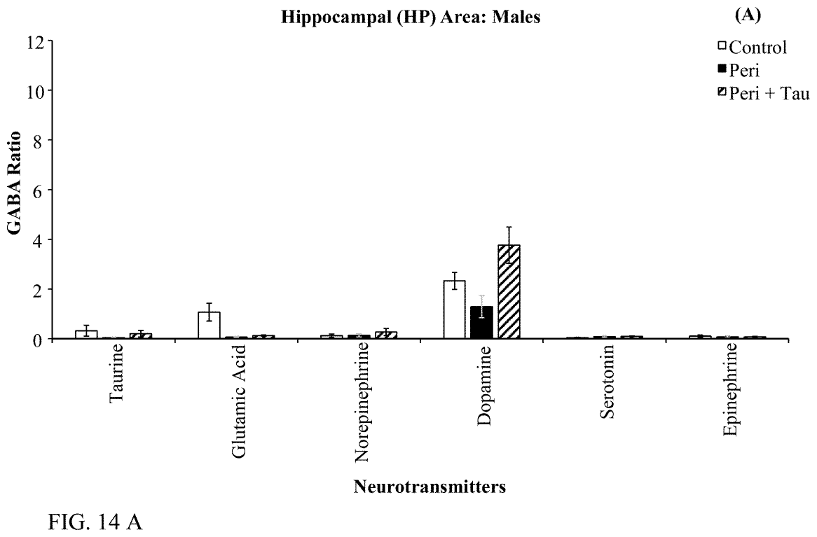

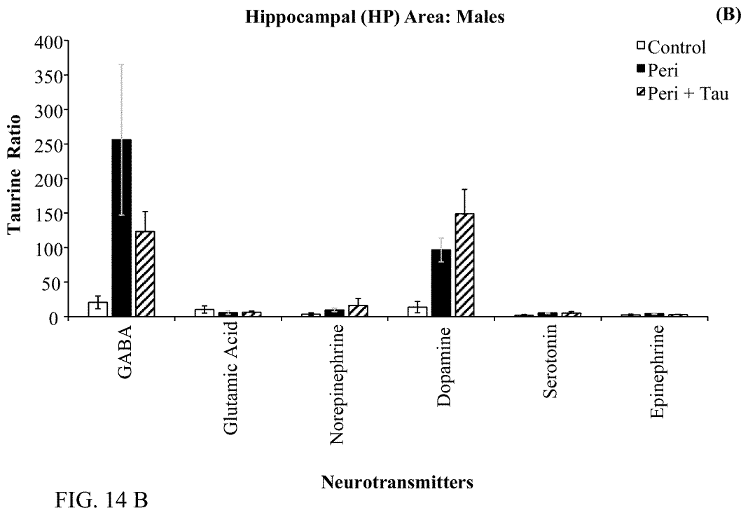

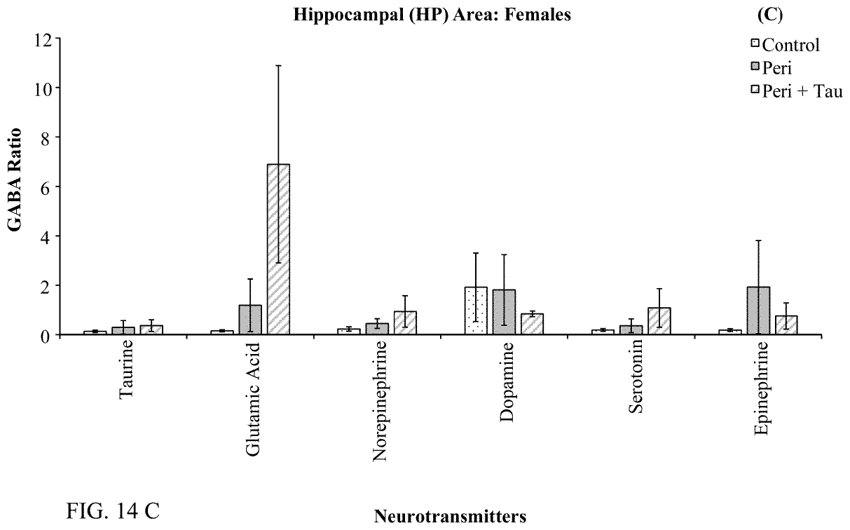

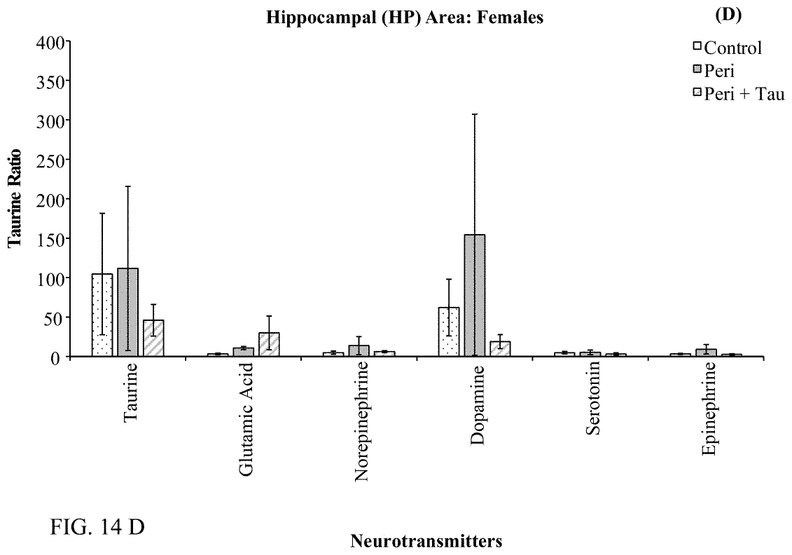

FIGS. 14A-14D illustrates the male (FIG. 14A & FIG. 14B) and female (FIG. 14C & FIG. 14D) rats LC/MS GABA:Neurotransmitter (FIG. 14A & FIG. 14C) and Taurine:Neurotransmitter (FIG. 14B & FIG. 14D) ratios in the hippocampal (HP) areas that regulate learning and memory. The data reveal that in males the GABA:Dompamine ratios are reduced and the PrL in response to Pb²⁺-exposure, and female HP are less affected (FIG. 14A & FIG. 14C). In female rats, the GABA:Glutamic acid ratio is elevated in response to taurine co-treatment (FIG. 14C). In contrast, perinatal Pb²⁺-exposure elevated the Taurine:GABA ratio in male HP and not in females (FIG. 14B & FIG. 14D), whereas the female rates showed no differences in response to Pb²⁺-exposure or taurine. The data suggest that Pb²⁺-exposure alters GABA:Glutamate, GABA:Dopamine, and GABA:Taurine hippocampal signaling at the neurochemical level, which could reduce learning and memory states at the behavioral level.

The present study showed that developmental Pb²⁺-exposure caused significant frontoexecutive dysfunctions that persisted later in life when the mature rats were tested in the ASST. Further, these frontoexecutive dysfunctions are consistent with an environmentally induced developmental neuropathological disorder in response to a neurotoxicant such as Pb²⁺. The changes observed in the rat during the ASST on the behavior level corroborated with frontoexecutive altered neurochemical signaling across the PrL, IL, OV, and OVL, as well as, HP signaling as it related to learning and memory. Developmental Pb²⁺-exposure has been reported to cause changes in the expression of adrenergic and dopaminergic receptors in the forebrain and striatum of rats (Rossouw et al., 1987) and chronic Pb²⁺-exposure has been shown to differentially affect dopamine synthesis across brain regions (Jason & Kellog, 1981; Lucchi et al., 1980; Govoni et al., 1979), as well as, glutamatergic and GABAergic altered brain excitability balancing (Struzyiiska & Sulkowski, 2004). In a clinical case study of chronic Pb²⁺-exposed patients in Saudi Arabia, they found in their blood plasma levels elevated GABA, 5-HT, and DA with associated autism diagnoses when compared to healthy age-matched controls (EI-Ansary et al., 2011). Moreover, prior reports have also eluded to the potential overlap between autism and environmental Pb²⁺-exposure as a subset of childhood case studies exhibiting autism or autism developmental symptoms that could be assessed via neuropsychological testing (Lidsky & Schneider, 2005). Consistent with these clinical reports, prior studies regarding the molecular changes observed in response to developmental Pb²⁺-exposure and its translation with the behavioral and cognitive system levels have been shown to disrupt inhibitory learning with observed increases in impulsivity under fixed-interval of scheduled behaviors (Cory-Slechta et al., 1998). Moreover, these findings were also shown to corroborate with Pb²⁺-induced learning impairments because of changes to the dopaminergic, cholinergic, and glutamatergic neurotransmitter systems (Cory-Slechta, 1995). Thus, the effects of low-level developmental Pb²⁺-exposure can significantly affect dopaminergic systems that provide incentive, motivation, mood balancing, along with other neurotransmitter systems that permit heighted arousal states in which to cognitively engage with one's environment. Further, it is suggested that through such a psychological profile, one could benefit from psychotropic medication that could prevent frontoexecutive dysfunction by regulating directly or indirectly dopamine tone in the frontal lobes.

The data obtained from the present study are in agreement with the findings from earlier reports. Thus, Pb²⁺-exposure appears to effect a cluster of neurotransmitter systems differentially across neurodevelopment in a sex-specific manner. Earlier reports on developmental Pb²⁺-neurotoxicity restricted their reports to one sex, thereby limiting comparative analyses as those produced herein. Further, taurine was shown to be effective in mitigating or at least reducing most of the Pb²⁺-induced frontoexecutive dysfunctions that were observed in the Perinatal rats. Developmental Pb²⁺-exposure also caused sex-based differences in the ASST performance that were far less dysfunctional following taurine co-treatment with distinct improvements in working memory and reacquisition learning performance, and more focused learning performances with less errors. Thus, taurine may provide a wide-range of neuroprotection within and across the neuro-developmental signaling pathways that later govern frontoexecutive functions. Consistent with prior reports, taurine may serve to prevent brain excitability, by balancing the GABA-shift in early development (Ben-Ari, 2002; Ben-Ari et al., 2012) and ensuring an adequate level of neurotransmitter tone across the establishment and maintenance of neurochemical signaling (Chan et. al., 2014), emotional and age-dependent signaling (Neuwirth et al., 2013; Neuwirth et al., 2015). Further studies have also show taurine's role in contributing to the regulation of context-dependent goal-directed behaviors (Neuwirth et al., 2013; Neuwirth, 2014; Neuwirth et al., 2017; Neuwirth et al., 2019a), with no apparent adverse effects on locomotor activity or anxiety behaviors (Santora et al, 2013; El Idrissi et al., 2011; El Idrissi et al. 2009). Thus, taurine may prove useful as a psychopharmacotherapy for treating or counteracting against neurotoxicants such as Pb²⁺.

In summary, this study shows that perinatal Pb²⁺-exposure can cause frontoexecutive dysfunctions in the rat model that persists across the lifespan. These frontoexecutive dysfunctions effect males and females in a sex-dependent manner, which require further study. Moreover, the sex-dependent neuropsychological profiles could be observed at both the behavioral (i.e., in the ASST) and the neurochemical levels (i.e., LC/MS data). Although, individual rat differences in frontoexecutive dysfunction could be observed, group differences were also observed in this study, thereby suggesting that the ASST is sensitive in revealing frontoexecutive dysfunction at the behavioral level in rats. This is significant as most reports on low-level Pb²⁺-exposure historically shows reduced sensitivity at the behavioral level for showing significant hippocampal learning deficits. Thus, perhaps frontoexecutive behavioral tests of attentional mechanism may prove more useful than hippocampal test in revealing a fine-grained analysis of Pb²⁺-impacts during neurodevelopment. Further, taurine co-treatment revealed a sex-dependent recovery in the rats exposed to perinatal Pb²⁺-exposure. Therefore, Pb²⁺ has been shown to disrupt GABAergic mediated networks that are, in part, responsible for regulating emotional-dependent learning and memory behaviors, and less is known regarding its involvement in frontoexecutive functions (Neuwirth et al., 2019a). Thus, this study presents a case for considering taurine as a psychopharmacotherapy for treating neurodevelopmental Pb²⁺-exposure as a means to improve one's frontoexecutive functions across their lifespan. The present study serves to open a new dialogue for clinical trials to consider using taurine therapy in treating Pb²⁺-exposed children that remain in environments that remain Pb²⁺-contaminated (Neuwirth, et al., 2018b).

### Example 2—Assessing the Anxiolytic Properties of Taurine_Derived Compounds in Rats Following Developmental Lead Exposure: A Neurodevelopmental and Behavioral Pharmacological Study

Lead (Pb²⁺) is a developmental neurotoxicant that causes alterations in the brain's excitation-to-inhibition (E/I) balance. By increasing chloride concentration through GABA-_{AR}, taurine serves as an effective inhibitory compound for maintaining appropriate levels of brain excitability. Considering this pharmacological mechanism of taurine facilitated inhibition through the GABA-_{AR}, the present study sought to explore the anxiolytic potential of taurine derivatives. Treatment groups consisted of the following developmental Pb²⁺-exposures: Control (0 ppm) and Perinatal (150 ppm or 1,000 ppm Pb²⁺ acetate in the drinking water). Rats were scheduled for behavioral tests between postnatal days (PND) 36-45 with random assignments to either solutions of Saline, Taurine, or Taurine Derived compound (TD-101, TD-102, or TD-103) to assess rats' responsiveness to each drug in mitigating the developmental Pb²⁺-exposure through the GABAergic system. Long Evans Hooded rats were assessed using an Open Field (OF) test for preliminary locomotor assessment. Approximately 24-hrs after the OF, the same rats were exposed to the Elevated Plus Maze (EPM) and were given an i.p. injection of 43 mg/Kg of the Saline, Taurine, or TD drugs 15-min prior to testing. Each rat was tested using the random assignment method for each pharmacological condition, which was conducted using a triple-blind procedure. The OF data revealed that locomotor activity was unaffected by Pb²⁺-exposure with no gender differences observed. However, Pb²⁺-exposure induced an anxiogenic response in the EPM, which interestingly, was ameliorated in a gender-specific manner in response to taurine and TD drugs. Female rats exhibited more anxiogenic behavior than the male rats; and as such, exhibited a greater degree of anxiety that were recovered in response to Taurine and its derivatives as a drug therapy. The results from the present psychopharmacological study suggests that Taurine and its derivatives could provide useful data for further exploring the pharmacological mechanisms and actions of Taurine and the associated GABAergic receptor properties by which these compounds alleviate anxiety as a potential behavioral pharmacotherapy.

The present study sought to build upon prior reports in which develop-mental Pb²⁺-exposure induced E/i imbalances that caused learning and memory deficits and were recovered by acute taurine treatment through the GABA-_{AR} system (Neuwirth, 2014; Neuwirth et al., 2017; Neuwirth, 2018). Early disruption of the brain's E/I balancing between the Glutamatergic (i.e., excitatory) and GABAergic (i.e., inhibitory) systems have been consistently identified as a contributing neurodevelopmental risk factor for seizure and other closely related neuropathologies (Ben-Ari, 2002; Ben-Ari et al., 2012). Taurine has been increasingly shown to mitigate against brain E/i imbalances in animal models of epilepsy (El Idrissi et al., 2003) through upregulation of glutamic acid decarboxylase (GAD) and interactions with the GABA-_{AR} B2/B3 subunits (L'Amoreaux et al., 2010). In addition, other reports have shown that taurine has been neuroprotective by sustaining GABAergic signaling during senescence where, on the other developmental continuum, the E/I balance begins to weaken with age (El Idrissi et al., 2013) with evidence supporting cognitive improvement in learning (El Idrissi, 2008; Neuwirth et al., 2013) and motor abilities (Santora et al., 2013) of aged animals.

In addition to taurine pharmacological therapy, the present study evaluated the effects of developmental Pb²⁺-toxicity on locomotion and anxiety, which are partially regulated by the GABAergic system. Consistent with previous reports (Neuwirth et al., 2017; Neuwirth, 2014), the present study explored whether the acute administration of taurine and taurine derivatives would recover the Pb²⁺-induced neurobehavioral aberrations in the rat model. Furthermore, the present study sought to evaluate gender-based differences in Pb²⁺ vulnerabilities and taurine as well as taurine derivatives to recover gender-specific alterations of the GABAergic mediated behaviors. Lastly, Pb²⁺-dosage was examined to determine the extent of GABAergic dysfunctions that could be assessed by their functionally associated behaviors in response to developmental Pb²⁺-exposure and the potential for taurine as well as taurine derivatives as a psychopharmacological treatment options for low-level Pb²⁺-exposures (i.e., ≤39 μg/dL) as a pilot study.

Methods

In accordance with The SUNY Old Westbury (SUNY-OW) IACUC approval guidelines, Long-Evans Norwegian hooded male (N=10) and female rats (N=20) (Taconic, N.J.) were paired for breeding and their male F1 generation were used for future experimentation. Rat litters were culled to 8-10 pups in order to control for maternal social influences on neurodevelopmental and behavioral outcomes that would be studied in later development. All rats were fed regularly with Purina rat chow (RHM1000 #5P07) ad libitum. However, control rats were provided regular water, while the experimental rats were fed water containing Pb²⁺ acetate (Sigma Aldrich, St. Louis, Mo.) from pairing throughout gestation and continued through weaning at postnatal day (PND) 22 (i.e., constituting a Perinatal Pb²⁺ developmental exposure model). At PND 22 Pb²⁺-exposures ceased and all rats returned to a regular water regimen. Rats assigned to the Peri-22 150 ppm group (drank a Pb²⁺ acetate water of [363.83 μM]) and the Peri-22 1,000 ppm group (drank a Pb²⁺ acetate water of [2.43 mM]) and all treatments were administered ad libitum. Prior to behavioral testing, all rats were handled for 10-min per day for 1-week. Between PND 36-45 rats were assigned the open field test and 24-hrs later, the elevated plus maze test.

Blood Pb²⁺-Level Analyses

At PND 22 immediately following the end of Pb²⁺ exposure, a separate group of male and female rats (i.e., with a representative sample culled from litter) were sacrificed (n=4 per gender, per Peri-22 150 ppm and Pe-ri-22 1,000 ppm treatment group) and their blood samples were collected and analyzed consistent with previous reports (Neuwirth, 2014; Neuwirth et al., 2017, Neuwirth et al., 2018). Briefly, blood samples were collected within 2 mL anti-coagulant ethylenediaminetetraacetic acid (EDTA) coated syringes (Sardstedt, Germany), mixed to prevent coagulation, and then frozen at −80° C. Blood samples were analyzed using a commercial ESA LeadCare II Blood Lead Analyzer system (Magellan Diagnostics, North Billerica, Mass.) to determine the amount of Pb²⁺ in the blood by electro-chemical anodic stripping voltammetry (ASV) to eliminate any potential for experimenter bias. The ASV method was conducted by taking 50 μL of whole blood mixed with 250 μL of hydrochloric acid solution (0.34 M) and then applying the final mixture to the lead sensor strip and inserted in- to the ESA LeadCare II Blood Lead Analyzer system to determine BLLs. After 3 minutes, the BLLs were reported from the instrument in μg/dL with a lower sensitivity cut off value of 3 μg/dL and a high sensitivity cut off value of 65 μg/dL (i.e., SEM±1.5 μg/dL sensitivity detection level).

The Open Field Test

Between PND days 36-45 a series of naivë rats from the F1 generation offspring (N=159) comprised of both males (n=80) and females (n=79) were subjected to an open field test (OF). The treatment groups were as follows: Control males (n=30), Peri-22150 ppm Pb²⁺ males (n=32), and Peri-221,000 ppm Pb²⁺ males. Control females (n=18), Peri-22 150 ppm Pb²⁺ females (n=30), and Peri-22 1,000 ppm Pb²⁺ females (n=19), respectively. All rats were examined during 10-min of locomotor exploration in the OF apparatus (376 mm H×914 mm W×615 mm L) in a dark room illuminated with red lighting (30 Lux) to promote locomotor activity in order to assess any motor disruption as a consequence of Pb²⁺-exposure. Locomotor variables included Total Distance Traveled measured in meters (m) and Overall Average Speed measured in meters/second (m/s).

Taurine and Taurine Derivative Drug Preparations and the Elevated Plus Maze Test

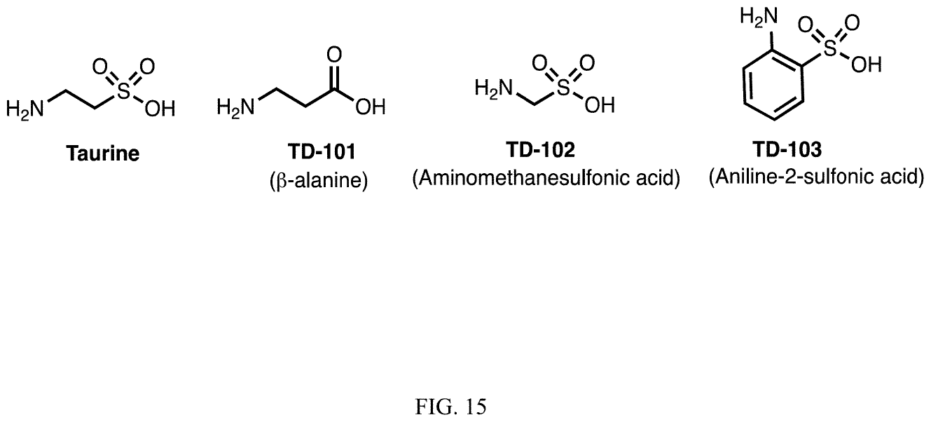

The next day following the OF assessment, the male and female rats were randomly assigned to one of six Dug treatment conditions (i.e., No Drug, Saline, Taurine (NH₂CH₂CH₂SO₃H-FW: 125.15 g/mol) (Sigma Aldrich, St. Louis, Mo.), Taurine Derivative (TD)-101 (C₃H₇NO₂ 89.09 g/mol), TD-102 (CH₅NO₃S 111.12 g/mol), or TD-103 (C₆H₇NO₃S 173.19 g/mol), respectively (see FIG. 15). All Taurine and TD compounds were dissolved in physiological buffered saline (PBS) with a pH of 7.4 as a final systemic concentration of [10 mM] and were then sterilized by syringe filtration (0.2 μm) prior to being administered.

Males were assigned as follows: No Drug (n=20), Saline (n=11), Taurine (n=13), TD-101 (n=14), TD-102 (n=11), and TD-103 (n=14). Females were assigned as follows: No Drug (n=17), Saline (n=10), Taurine (n=11), TD-101 (n=11), TD-102 (n=13), and TD-103 (n=13), respectively. Rats were administered their randomly assigned drug treatment as a triple-blind procedure via i.p. injection 15-min prior to EPM testing. Drugs were administered as equivalent 43 mg/kg drug injections (i.e., to standardized against Taurine as a reference) across all treatments to draw appropriate comparative outcomes. All rats were examined during 10-min of anxiety-like behavioral assessments in the EPM. The EPM apparatus (external dimensions: 800.1 mm H×1,104.9 mm W×1,104.9 mm L; closed arm dimensions: 101.6 mm W×1,104.9 mmL×304.8 mm H walls; open arm dimensions: 101.6 mm W×1,104.9 mm L; the platform was elevated off the floor by 495.3 mm H) was within a brightly illuminated room (300 Lux) to promote an anxiogenic response. The anxiogenic behaviors were evaluated in order to assess the effects of Pb²⁺-exposure to evoke anxiety-like behaviors and the potential for Taurine and TDs treatments to provide anxiolytic pharmacotherapy within the EPM. Anxiety-like behavioral variables included the Open-to-Closed (OTC) Ratio and a representative group mean heat plot to assess activity across the 10-min of the EPM.

Data Analyses

Data were recorded in real-time and analyzed using the Anymaze® video tracking software (Stoelting Co., Wood Dale, Ill.) transmitted via a ceiling mounted Logitech C310 Hi-speed USB 2.0 web camera (High-definition video with 1,280×720 pixels and 5 MP photo quality). The web camera was relayed to a standard Dell D16M Inspiron 3847 Desktop computer equipped with Windows 10 64-bit operating systems, 8 GB Dual Channel DDR3 1,600 MHZ (4 GB×2), 1 TB 7,200 PRM Hard Drive, and a 4^{th} Generation Intel® Core™ i3-4170 Processor (3 M Cache, 3.70 GHz), and displayed through a Dell 20″ E2016H monitor with an optimal resolution of 1,600×900 pixels at 60 Hz. Data were recorded as digital videos that were analyzed using AnyMaze® software. Animal tracking was based on contrast relative to background. Different zones were labeled and indicated on the monitor for both the OF and EPM. Three tracking points were specified one on the rat's head, the center of the rat's body, and the rat's tail. A Microsoft Excel spreadsheet was generated containing all the parameters specified for both the OF and EPM tests, respectively.

Statistical Analyses

All behavioral data were collated in Microsoft Excel and later analyzed in IBM SPSS V. 24 (IBM, Inc. Armonk, N.Y.). For the OF tests, a Repeated Measures ANOVA was conducted using Time and Pb²⁺ Exposure as the within subjects factors and Pb²⁺ Exposure as the between-subjects factors for the dependent variables of Total Distance Travelled (meters) and Overall Average Speed (meters/second). For the EPM tests, a Multi-Factorial ANOVA with Treatment and PPM as fixed-factors was used to evaluate the dependent variables of the OTC and Drug Treatment Condition. The criteria for significance was set at α=0.05% with a 95%±SEM. Significant differences were determined by an unequal Tukey's HSD post hoc multiple comparisons tests along with a partial Eta-square (ηₚ²) for determining effect sizes where applicable.

Results

BLLs as a Function of Pb²⁺-Dose and -Exposure Cessation Prior to Behavioral Testing.

A separate set of rats was used to determine BLLs (n=4 males and n=4 females for both the Peri-22 150 ppm and Peri-22 1,000 ppm Treatment groups). Rats were sacrificed at PND 22 when their Pb²⁺ exposure was stopped. Animals were then anesthetized with 50 mg/kg of sodium pentobarbital via i.p. injection, and once non-reflexive, a cardiothoracic blood draw was taken and analyzed with the LeadCare II system as stated above. The results showed no differences in BLL as a function of gender. Each Pb²⁺-treatment at the time of sample collection resulted in BLLs ranging from 3.3-10.7 μg/dL (SD±1.57) for Peri-22 150 ppm rats (p<0.001***) and from 9.0-17.8 μg/dL (SD±2.86) for Peri-22 1,000 ppm (p<0.001***), respectively. The control rats were Pb²⁺ negative. Thus, the BLL samples obtained in this study were less than the 39 μg/dL chelation therapy limit. The BLL samples from the behaviorally tested rats were al-so drawn at PND 55 days following the conclusion of the study; however, their BLLs were below the 3.3 μg/dL detection limit. The reduction in circulatory BLLs would be a combination of clearing from the body as well as bodily tissue absorption of Pb²⁺ in the blood. This is consistent with reports from the U.S. Agency for Toxic Substances and Disease Registry (ATSDR, 2007) that Pb²⁺ is not uniformly distributed in bone, blood, and soft mineralizing tissues; thus, requiring careful medical management in children.

Developmental Pb²*-Exposure Showed No Difference in Locomotor Activity Irrespective of Pb²⁺-Dose or Gender.

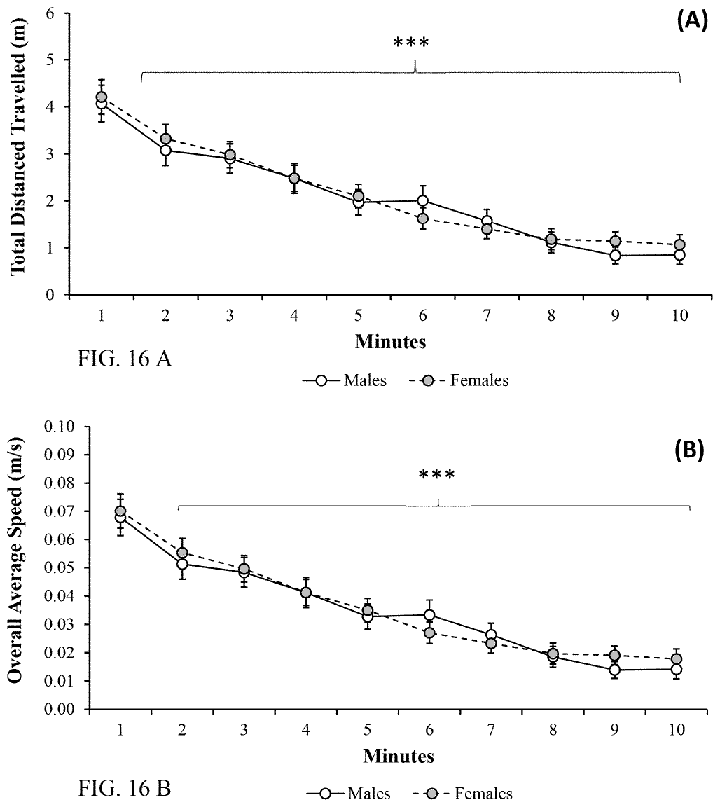

The OF was used as a preliminary assessment for locomotor disruption to evaluate the potential for any confounding behavioral effects that might influence the interpretation of anxiogenic and anxiolytic behaviors within the subsequent EPM test. As such, a preliminary locomotor assessment was first conducted to determine whether there were any gender-based differences in the OF. The within-subject factors for the Total Distance Travelled (m) assessment showed a significant effect of Time F_{(9,58)}=81.125, p<0.001***, (ηₚ²)=0.583 (FIG. 16A), but no significant Time×Gender interaction F_{(1,58)}=0.771, p=0.563 n/s (FIG. 16A). The between-subjects assessment of Total Distance Travelled (m) was also not significant F₍₁₎=0.41, p=0.839 n/s (FIG. 16A). The rats Overall Average Speed (m/s) was also assessed, and the within-subject factors revealed a significant effect of Time F_{(9,58)}=78.992, p<0.001*** (FIG. 16B), (ηₚ²)=0.577, but no significant Time×Gender interaction was observed F_{(1,58)}=0.633, p=0.671 n/s (FIG. 16B). The between-subjects assessment of Overall Average Speed (m/s) was also not significant F₍₁₎=0.069, p=0.793 n/s (FIG. 16B).

Referring to FIGS. 16A and 16B, preliminary assessment of rat locomotor activity in the OF as an effect of Gender (males=open circles; females=grey circles). Data show for both Total Distance Travelled (m) (FIG. 16A) and Overall Average Speed (m/s) (FIG. 16B), that there were no significant differences in rat locomotor activity in the OF as a function of Gender. However, as a function of Time, there was a significant effect across the 10-min of the OF in which the rats gradually shift from high-to-low locomotor activity as they habituated to the OF (p<0.001***). Thus, indicating that both rat Genders are equal in their locomotor behavioral profiles.

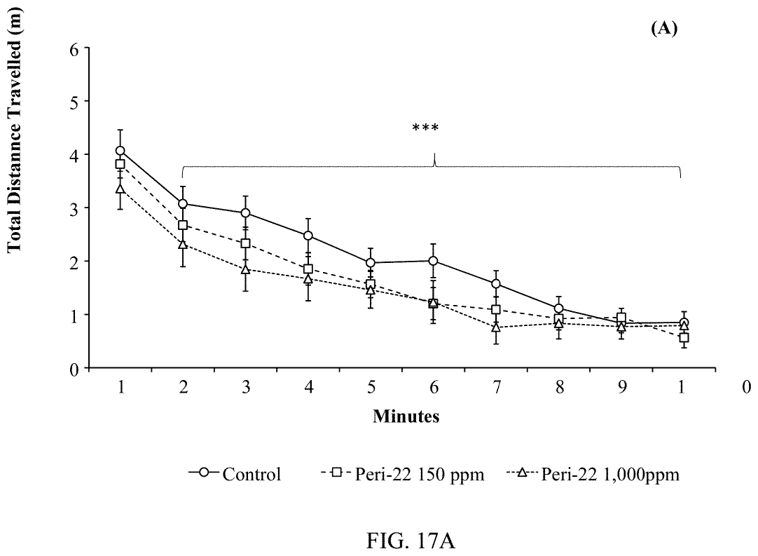

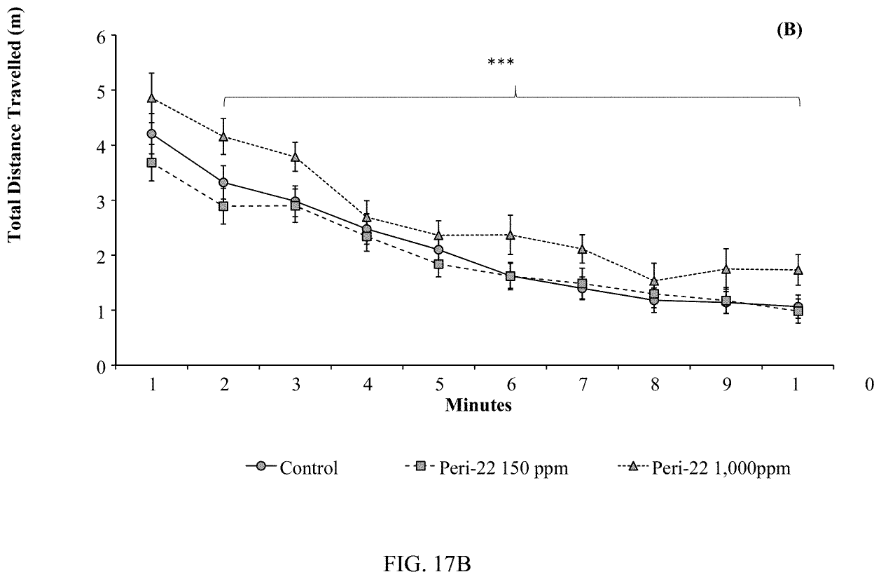

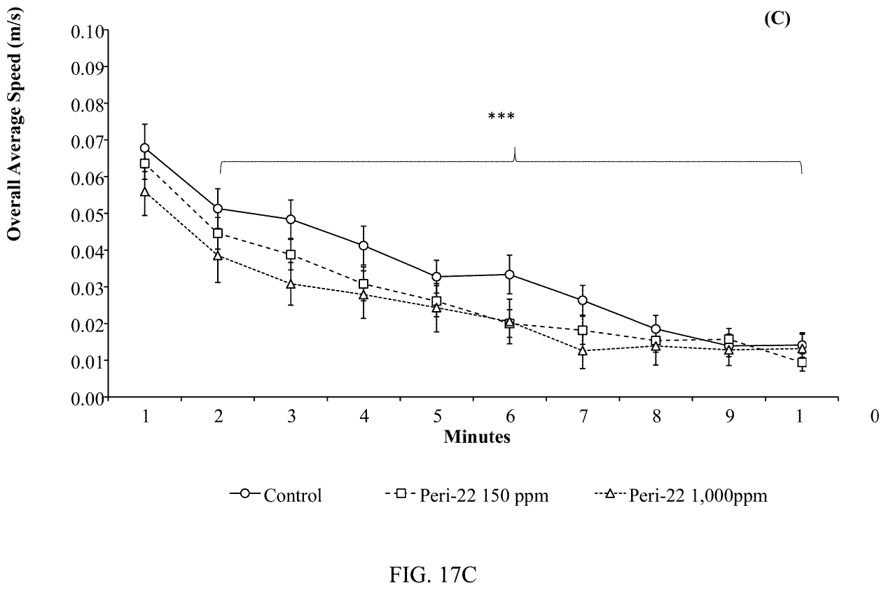

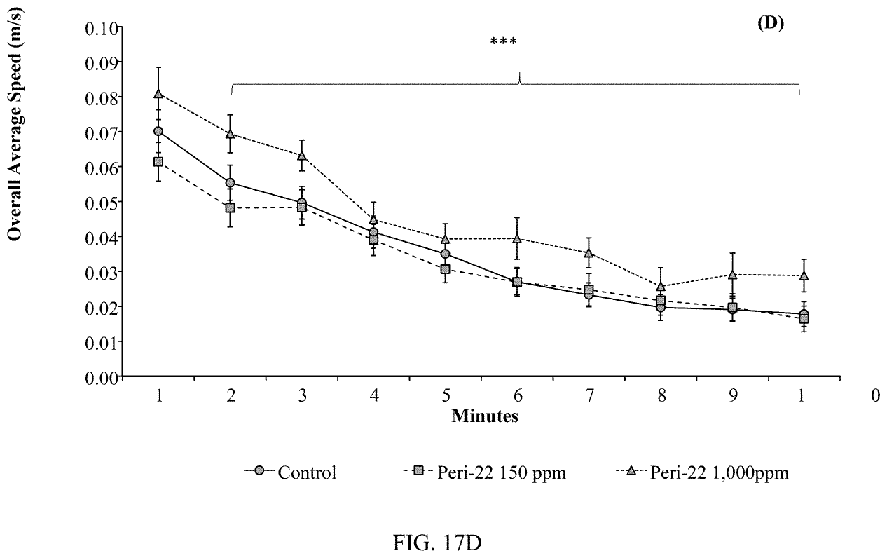

Following the gender-based preliminary assessment of locomotor activity, each gender was separately examined to determine whether any within-gender effects were observed as a function of 150 ppm and 1,000 ppm Pb²⁺-exposures. For the OF assessment of Total Distance Travelled (m) in male rats, the within-subject factors revealed a significant effect of Time F_{(9,77)}=79.136, p<0.001***, (ηₚ²)=0.07 (FIG. 17A), but there was no significant Time×Pb²⁺ Exposure interaction F_{(2,77)}=1.112,p=0.349 n/s (FIG. 17A). The between-subjects assessment of Total Distance Travelled (m) was also not significant F₍₂₎=1.694, p=0.190 n/s (FIG. 17A). In addition, the Overall Average Speed (m/s) was assessed in female rats and the within-subject factors revealed a significant effect of Time F_{(9,77)}=79.174, p<0.001, (ηₚ²)=0.507 (FIG. 17C), but there was no significant Time×Pb²⁺ Exposure interaction F_{(2,77)}=1.115, p=0.347 n/s (FIG. 17C). The be-tween-subjects assessment of Overall Average Speed (m/s) was also not significant F₍₂₎=1.698, p=0.190 n/s (FIG. 17C). In contrast, the OF assessment of Total Distance Travelled (m) in fe-male rats, the within-subject factors revealed a significant effect of Time F_{(9,76)}=90.058, p<0.001***, (ηₚ²)=0.542 (FIG. 17B), but there was no significant Time×Pb²⁺ Exposure interaction F_{(2,76)}=0.947, p=0.482 n/s (FIG. 17B). The between-subjects assessment of Total Distance Travelled (m) was also not significant F₍₂₎=2.471, p=0.091 n/s (FIG. 17B). In addition, the Overall Average Speed (m/s) was assessed in female rats and the within-subject factors revealed a significant effect of Time F_{(9,76)}=88.481, p<0.001, (ηₚ²)=0.538 (FIG. 17D), but there was no significant Time×Pb²⁺ Exposure interaction F_{(2,76)}=1.042, p=0.405 n/s (FIG. 17D). The be-tween-subjects assessment of Overall Average Speed (m/s) was also not significant F₍₂₎=2.449, p=0.093 n/s (FIG. 17D).

Referring now to FIGS. 17A-17D, FIGS. 17A-17D shows an assessment of Pb²⁺-exposure (150 ppm=squares; 1,000 ppm=triangles) on rat locomotor activity in the OF and its influences within-Gender (males=open symbols; females=grey symbols). Data show for both Total Distance Travelled (m) (FIGS. 17A & 17B) and Overall Average Speed (m/s) (FIGS. 17C & 17D), that there were no significant differences in locomotor activity in the OF as a function of Pb²⁺ exposure nor Gender. However, as a function of Time, there was a significant effect across the 10-min of the OF in which the rats gradually shift from high-to-low locomotor activity as they habituate to the OF (p<0.001***). Thus, indicating that both rat Genders were not influenced by Pb²⁺-exposure in their locomotor behavioral profiles.

Developmental Pb²⁺-Exposure Induced Gender-Based Differences in Anxiogenic Behaviors that were Recovered by Taurine and Taurine Derivative Anxiolytic Drug Treatments.

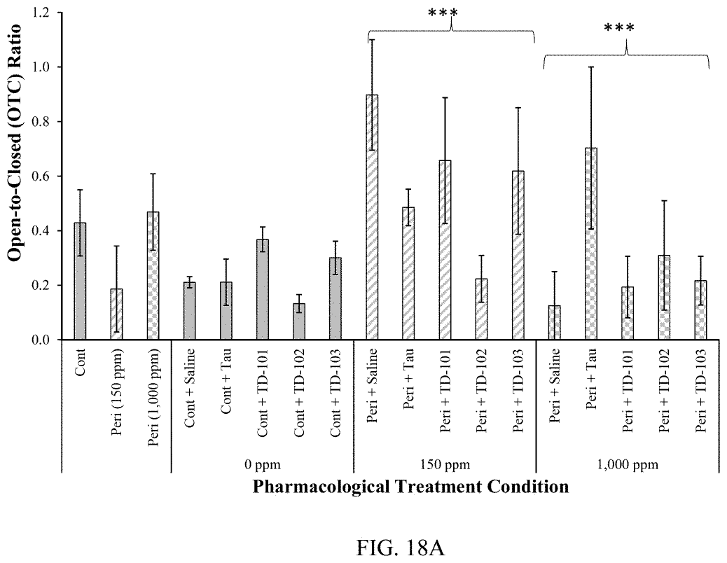

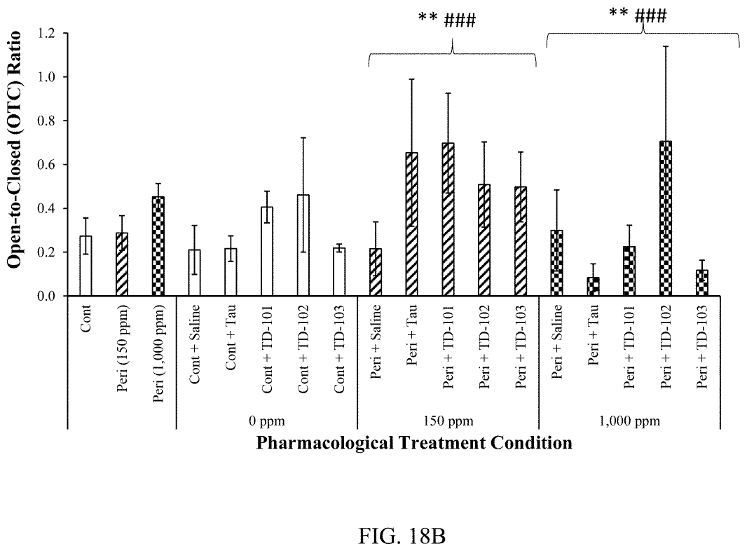

After 24 hrs following the OF, rats were subjected to the EPM to compare the within-gender differences in response to both developmental Pb²⁺-exposure as a function of PPM and Drug Treatment Condition effects on the OTC ratio. Male rats showed no significant effect of Treatment for the OTC ratio F₍₁₎=1.177, p=0.282 n/s (FIG. 18A), yet revealed a significant effect of Treatment and PPM for the OTC ratio F_{(1,2)}=153.452, p<0.001***, (ηₚ²)=0.684 (FIG. 18A). Interestingly, despite these Pb²⁺-induced differences, male rats showed no significant effects of Drug Treatment Condition for the OTC ratio F₍₅₎=0.673, p=0.645 n/s (FIG. 18A), nor any significant effect on Drug Treatment Condition and PPM for the OTC ratio F_{(5,3)}=0.014, p=1.000 n/s (FIG. 18A). Also, male rats exhibited no significant Treatment×Drug Treatment Condition interaction for the OTC ratio F_{(1,2)}=0.043, p=0.999 n/s (FIG. 18A), nor any significant Treatment×Drug Treatment Condition×PPM interaction for the OTC ratio F_{(1,2,5)}=0.014, p=1.000 n/s (FIG. 18A). In contrast, female rats showed no significant effect of Treatment for the OTC ratio F₍₁₎=0.168, p=0.683 n/s (FIG. 18B), yet revealed a significant effect of Treatment and PPM for the OTC ratio F_{(1,2)}=10.017, p<0.01**, (ηₚ²)=0.124 (FIG. 18B). Remarkably, female rats exhibited Pb-induced anxiogenic differences, and showed more sensitivity to the Drug Treatment Conditions, when compared to male Pb²⁺ exposed rats. Specifically, female rats showed significant effects of Drug Treatment Condition for the OTC ratio F₍₅₎=2.951, p<0.05*, (ηₚ²)=0.077 (FIG. 18B), and a significant effect on Drug Treatment Condition and PPM for the OTC ratio F_{(5,3)}=14.659, p<0.001***, (ηₚ²)=0.292 (FIG. 18B). Furthermore, female rats exhibited a significant Treatment×PPM×Drug Treatment Condition interaction for the OTC ratio F_{(1,2,5)}=2.896, p<0.05*, (ηₚ²)=0.166 (FIG. 18B).

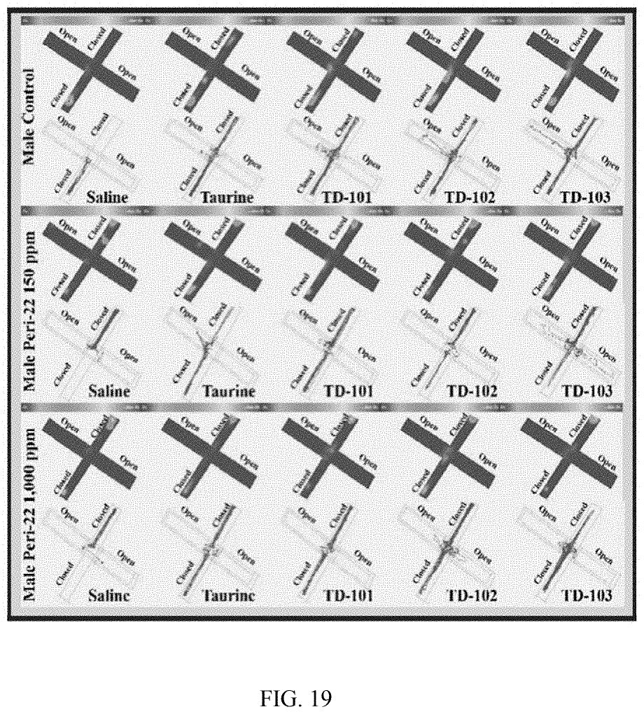

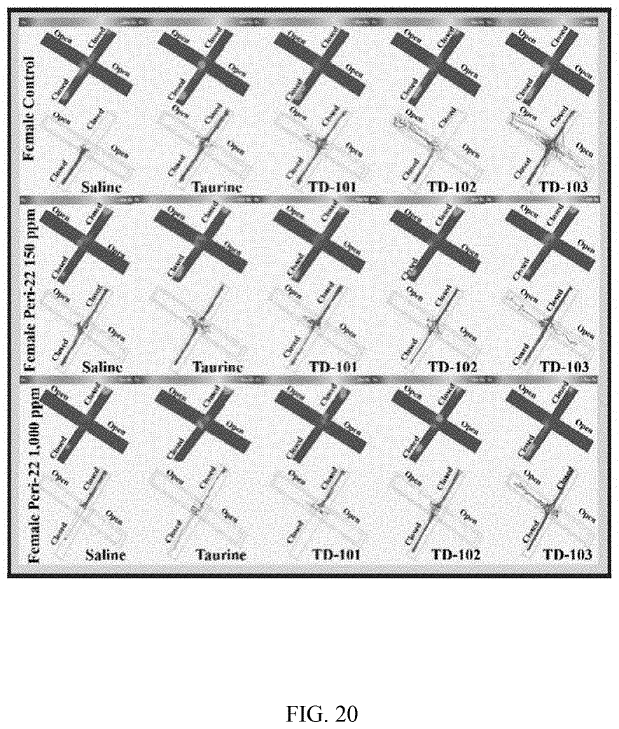

To further illustrate the EPM data, FIG. 19 (males) and FIG. 20 (females) shows a representative individual rat track plot in addition to the OTC ratio as a function of group mean activity during the 10-min of the EPM. Low activity (i.e., anxiogenic responses) can be visualized by the dark-blue inactive freezing responses. In contrast, high activity in the EPM (i.e., anxiolytic responses) can be visualized by the increase in color shades shifting from light blue to green, yellow, orange, and red activity responses.

Referring now to FIG. 18, effects of Pb²⁺-exposure (150 ppm=diagonal line bar pattern; 1,000 ppm=checkered bar pattern) on Open-to-Close (OTC) ratio in the EPM and its influences within-Gender (males=upper panel A; females=lower panel B). Data show for that there was an effect of PPM in both male rats (p<0.001***) and female rats (p<0.01**), respectively. However, male rats did not show a significant effect of Drug Treatment Condition, yet female rats did show a significant effect of Drug Treatment Condition (p<0.001^{###}). The data suggest that female rats were more responsive to Taurine and Taurine Derivative pharmacotherapy than male rats. However, through this pilot study, there was an emerging trend that dependent upon the amount of Pb²⁺-exposure (PPM) and gender, per-haps different taurine derivatives may prove to be useful in facilitating recovery of Pb²⁺-induced behavioral anxiety in the EPM.

Referring now to FIG. 19, a visual representation of an individual rat track plot from each treatment condition and their group mean activity average across the 10-min EPM test for male rats. Data are shown as a function of Pb²⁺-exposure (PPM; upper panel 0 ppm; middle panel 150 ppm; and lower panel 1,000 ppm). In addition, data are organized by Drug Treatment Condition vertically from left-to-right (Saline; Taurine; TD-101; TD-102; TD103). Control male rats show an increased anxiolytic response in the EPM to taurine and taurine derivatives. However, Pb²⁺-exposed rats show less sensitivity and selectivity to drug treatments with perhaps less potential for taurine and taurine derived pharmacotherapy.

Referring now to FIG. 20, a visual representation of an individual rat track plot and their group mean activity average across the 10-min EPM test for female rats. Data are shown as a function of Pb²⁺-exposure (PPM; upper panel 0 ppm; middle panel 150 ppm; and lower panel 1,000 ppm). In addition, data are organized by Drug Treatment Condition vertically from left-to-right (Saline; Taurine; TD-101; TD-102; TD103). Control female rats show an increased anxiolytic response in the EPM to taurine and taurine derivatives. Notably, Pb²⁺-exposed rats show both a sensitivity and selectivity to certain taurine derivatives with the potential for more anxiolytic effects than taurine.

The present study sought to examine the effects of developmental Pb²⁺-exposure on locomotor activity within the OF and anxiogenic behaviors within the EPM as a function of Treatment, Pb²⁺-dose (i.e., PPM), and Gender, as well as the pharmacological treatment by Taurine and Taurine Derived Drug Treatment Conditions. In the OF, no differences were observed in males or females with respect to the Total Distance Travelled (m) or the Overall Average Speed (m/s) as measures of locomotor activity. Furthermore, despite developmental Pb²⁺-exposure, no differences in any of these OF measures were observed. This suggests that at the 150 ppm and 1,000 ppm Perinatal exposure time-period of development in the Long Evans Hooded rat, the Pb²⁺-exposure produced no behavioral deficits or excesses that would have been deemed as abnormal locomotor activity. Thus, no evidence of issues with locomotor activity (i.e., that would have otherwise interfered with interpreting anxiety-like behaviors within the EPM) can be traced to the developmental Pb²⁺-exposure from the OF preliminary assessment.

In the EPM, the within-gender effects were assessed for anxiogenic behaviors that are evoked by the EPM testing apparatus and bright lighting effects. Female rats were observed to be more sensitive to the EPM and exhibited less OTC ratios when compared to males in the control treatment conditions. However, when comparing the within-gender effects as a function of Pb²⁺-exposure, male rats showed no differences in their OTC ratios, when compared to control males. In comparison, female rats that were exposed to Pb²⁺ also showed no differences in their OTC ratio relative to control females. Thus, it would appear that Pb²⁺ causes no anxiogenic behaviors in the EPM. However, the OTC ratio is a different dependent measure that is arguably more sensitive to drug effects in the EPM (Waif & Frye, 2007). As such, it assesses the reduction or anxiolytic properties of the rat's exploratory behavior to inhibit fear and approach the open arms more than the closed arms. Traditional EPM dependent measures, such as Time in the Closed Arm or Time in the Open Arm, are fair indicators of anxiogenic behaviors, but require careful interpretation. First, most studies using the EPM may only report one of these dependent measures, which only describe half of the anxiogenic profile of the animal model under study. Second, because the rats could be moving freely or freezing, “Time” alone is an insufficient descriptor of animal's behavior. Thus, unless clearly operationally defined, “Time” variable offers more obscurity than one would hope in EPM analyses. Lastly, the traditional EPM values do prove informative when carefully examined, operationalized, and interpreted.

The present study, sought to assess the effectiveness of Taurine and its derivatives in Drug Treatment Conditions for anxiolytic behavioral pharmacological effects on rats in the EPM. In this context, the OTC ratio served as a very sensitive dependent measure as it targets the increase in activity into the open arms relative to the activity into the close arms. Higher OTC ratio results in more anxiolytic the rat's behavioral response.

Conversely, the lower the OTC ratio the more anxiogenic the rat's behavioral response. The effects of the Drug Treatment Conditions revealed in this pilot study that the control male rats were most sensitive to Taurine Derivatives TD-101 and TD-102, whereas the control female rats were most sensitive to Taurine Derivatives TD-101 and TD-103. The Peri-22 150 ppm male rats seem to be sensitive and responsive to Taurine and each of the Taurine Derivative drugs, whereas Peri-22 1,000 ppm male rats were only sensitive to TD-102 in promoting anxiolytic OTC ratios. Remarkably, the Peri-22 150 ppm female rats showed sensitivity to Taurine and each of the Taurine Derivatives, except for TD-102; whereas Peri-22 1,000 ppm females were sensitive to Taurine and only TD-102.

These findings suggest that Pb²⁺-exposure perhaps changed the GABA-_{AR} subunit arrangement by altering the sensitivity to pharmacodynamic properties of the receptor activation states—that is functionally different in both a gender-specific manner and in response to the amount of Pb²⁺ endured in development. Furthermore, the type of direct neurotoxicant impact that Pb²⁺ inflicts upon the developing nervous system during critical stages of GABAergic neural development (Ben Ari, 2002, Ben-Ari et al., 2012; Neuwirth et al., 2018; Neuwirth et al., 2017; Neuwirth, 2014), could also alter the GABAergic tone and responsivity to GABAergic agonist drugs. The Taurine derivatives used in this study present a novel and, perhaps, a pioneering approach to the development and evaluation of new Taurine-like compounds that might foster more precise neuromodulatory actions of the GABA-_{AR} to counteract the neurotoxicant impacts of environmental Pb²⁺-exposure to the developing central nervous system.

In summary, this study shows that developmental Pb²⁺-exposure can have lifespan-lasting impacts on the central nervous system. In addition, based on the Taurine derivative used in this study, the amount of develop-mental Pb²⁺-exposure can, perhaps, influence the arrangement of the GABA-_{AR} in ways that alter its pharmacodynamics responsivity to GABA-_{AR} agonists. Furthermore, the chemical structure of the Taurine Derivatives provide new insights into examining specific drug treatments that might be uniquely matched to different Pb²⁺-exposure levels, and may be further customized to accommodate gender-specific needs given the different sensitivity to Taurine and Taurine Derived compounds through the EPM. Although, this study is limited to the EPM, future research may look to explore the effects of these Taurine Derivatives across a range of other behavioral test conditions to evaluate other cognitive and behavioral neurological conditions impacted by environmental Pb²⁺-exposure (see Ch. 70 Neuwirth et al., 2019). As such, this study paves the way for new re-search in investigating possible drug treatments that are safe, effective, and precisely match the underlying problems induced by neurotoxicants such as Pb²⁺. Future Pb²⁺ research should make a concerted effort to provide children with psychopharmacotherapies that may improve their quality of life across their lifespan; especially if they are unable to be removed for sources of environmental Pb²⁺-exposures.

### Example 3 (Prophetic Example)

A 3 year-old human subject having one or more neurological symptoms such as anxiety, loss of affection, or loss of cognitive function is presented to a physician with elevated Pb²⁺ levels above 30 μg/dL. The physician treats and ameliorates one or more neurological symptoms of Pb²⁺ poisoning by administering a therapeutically effective amount of taurine or taurine derivative to a subject in need thereof. The subject's presenting state is altered and is improved.

### Example 4 (Prophetic Example)

A physician treats a human subject suffering from symptoms of Pb²⁺ poisoning and presenting with one or more neurological symptoms. The physician administers a therapeutically effective amount of taurine or taurine derivative, or a pharmaceutical dosage form including taurine or taurine derivative to a subject in need thereof. The taurine, taurine derivative, or combinations thereof bind to one or more gamma amino butyric acid (GABA-_{A}) receptors, or gamma amino butyric acid (GABA-_{A}) receptor subunit configurations, to one or more glycine (Gly) receptors, or one or more glycine (Gly) receptors subunit configurations, to one or more n-methyl-D-aspartate (NMDA) receptors, or one or more n-methyl-D-aspartate (NMDA) receptors subunit configurations, and alter the state of the subject, wherein the subject's symptoms of Pb²⁺ poisoning improve.

### Example 5 (Prophetic Example)

A 4 year-old developing child (human) subject having one or more neurological symptoms such as anxiety, loss of affection, or loss of cognitive function is presented to a physician with elevated Pb²⁺ levels above 10 μg/dL. The physician treats and ameliorates one or more neurological symptoms of Pb²⁺ poisoning by administering a therapeutically effective amount of taurine or taurine derivative to a subject in need thereof. The subject's presenting state is altered and is improved. The physician also provides concurrent, or sequential chelation therapy to remove Pb²⁺ from the subject's blood. The therapeutically effective amount of taurine or taurine derivative is provided in a time-released pill or capsule dosage form.

Here and throughout the specification and claims, range limitations may be combined and/or interchanged, such ranges are identified and include all the sub-ranges contained therein unless context or language indicates otherwise.

The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

What is claimed:

1. A method of treating, ameliorating, or preventing one or more neurological symptoms of lead (Pb²⁺) poisoning in a subject having one or more neurological symptoms, comprising: administering a therapeutically effective amount of taurine or taurine derivative to a subject in need thereof.

2. The method of claim 1, the taurine or taurine derivative has a binding affinity sufficient to bind to one or more gamma amino butyric acid (GABA-_{A}) receptors, or one or more gamma amino butyric acid (GABA-_{A}) receptors subunit configurations.

3. The method of claim 1, wherein the taurine or taurine derivative has a binding affinity sufficient to bind to one or more glycine (Gly) receptors, or one or more glycine (Gly) receptors subunit configurations.

4. The method of claim 1, wherein the taurine or taurine derivative has a binding affinity sufficient to bind to one or more n-methyl-D-aspartate (NMDA) receptors, or one or more n-methyl-D-aspartate (NMDA) receptors subunit configurations.

5. The method of claim 1, wherein the subject comprises one or more n-methyl-D-aspartate (NMDA) receptors, wherein the taurine or taurine derivative has a binding affinity sufficient to bind the taurine or taurine derivative to the one or more n-methyl-D-aspartate (NMDA) receptor subunit configurations at one or more glycine binding sites.

6. The method of claim 1, wherein the taurine derivative is selected from the group consisting of a compound selected from the group consisting of 3-aminopropanoic acid, 2-aminobenzenesulfonic acid, 2-(aminoethyl)phosphonic acid, 3-amino-N-(trifluoromethyl)propenamide, 3-amino N-hydroxypropanamide, 2-aminoethane-1-sufinic acid, 3-aminopropane-1-sulfinic acid, 3-amino-3-fluoropanoic acid, 2-amino-2-fluoroethane-1-sulfinic acid, 3-amino-2-fluoropropane-1-sulfinic acid, 4-amino-3-fluorobutanoic acid, 3-amino-2-fluoropropanoic acid, 2-aminocyclopropane-1-carboxylic acid, and combinations thereof.

7. The method of claim 1, wherein the taurine or taurine derivative is a pharmaceutically acceptable salt, hydrate or solvate thereof.

8. The method of claim 1, wherein the taurine or taurine derivative is disposed within a pharmaceutically acceptable vehicle.

9. The method of claim 1, wherein the taurine or taurine derivative is administered during gestational, perinatal, and early postnatal development of the subject, and wherein the subject is exposed to lead (Pb²⁺).

10. The method or process of claim 1, wherein the taurine or taurine derivative is administered upon early maturation of the subject.

11. The method of claim 1, wherein the taurine or taurine derivative is administered through interperitoneal injection in quantities less than 43 mg/kg or through a second route of administration at equivalent physiological dosage.

12. The method of claim 1, wherein the taurine or taurine derivative is administered in a drinking water solution containing both lead (Pb²⁺) and taurine or taurine derivative, wherein the taurine or taurine derivative is present at about 0.05% of the total drinking water solution.

13. The method of claim 1, wherein the taurine or taurine derivative is administered in an extended release pill.

14. The method of claim 1, wherein the taurine or taurine derivative is administered intraperitoneal injection.

15. The method of claim 1, wherein the subject is a pregnant female mammal comprising a fetus, wherein the therapeutically effective amount is an amount sufficient for neuroprotection of the fetus from contact with lead (Pb²⁺).

16. The method of claim 1, wherein the subject is a developing child, wherein the therapeutically effective amount is an amount sufficient for neuroprotection of the child from contact with lead (Pb²⁺).

17. A composition for treating, ameliorating, or preventing one or more neurological symptoms of lead (Pb²⁺) poisoning in a subject, comprising:
a compound comprising one or more of: 2-aminoethane-1-sulfonic acid, 3-aminopropanoic acid, 2-aminobenzenesulfonic acid, 2-(aminoethyl)phosphonic acid, 3-amino-N-(trifluoromethyl)propenamide, 3-amino N-hydroxypropanamide, 2-aminoethane-1-sulfinic acid, 3-aminopropane-1-sulfinic acid, 3-amino-3-fluoropanoic acid, 2-amino-2-fluoroethane-1-sulfinic acid, 3-amino-2-fluoropropane-1-sulfinic acid, 4-amino-3-fluorobutanoic acid, 3-amino-2-fluoropropanoic acid, 2-aminocyclopropane-1-carboxylic acid, or a pharmaceutically acceptable salt, hydrate or solvate thereof.

18. The composition of claim 17, wherein the composition is disposed within a formulation comprising a pharmaceutically acceptable vehicle.

19. The composition of claim 18, wherein the formulation is an extended release composition or injectable solution.

20. A pharmaceutical formulation, comprising:
a compound selected from the group consisting of 2-aminoethane-1-sulfonic acid, 3-aminopropanoic acid, 2-aminobenzenesulfonic acid, 2-(aminoethyl)phosphonic acid, 3-amino-N-(trifluoromethyl)propenamide, 3-amino N-hydroxypropanamide, 2-aminoethane-1-sulfinic acid, 3-aminopropane-1-sulfinic acid, 3-amino-3-fluoropanoic acid, 2-amino-2-fluoroethane-1-sulfinic acid, 3-amino-2-fluoropropane-1-sulfinic acid, 4-amino-3-fluorobutanoic acid, 3-amino-2-fluoropropanoic acid, 2-aminocyclopropane-1-carboxylic acid, or a pharmaceutically acceptable salt, hydrate or solvate thereof; and a pharmaceutically acceptable vehicle, wherein the compound is present in an amount sufficient to bind to one or more gamma amino butyric acid (GABA-_{A}) receptors, one or more n-methyl-D-aspartate (NMDA) receptors, or one or more glycine (Gly) receptors disposed within a subject.
